(19) United States
(12) Patent Application Publication
(10) Pub. No.: US 2020/0023078 A1
(43) Pub. Date: Jan. 23, 2020

(54) ANIMAL MODEL SYSTEM AND USES THEREOF

(71) Applicants: Arizona Board of Regents on Behalf of the University of Arizona, Tucson, AZ (US); Ohio University, Athens, OH (US)

(72) Inventors: Magdalene So, Tucson, AZ (US); Jeffrey Frelinger, Tucson, AZ (US); Man Cheong Ma, Tucson, AZ (US); Daniel Powell, Tucson, AZ (US); Katherine Rhodes, Tucson, AZ (US); Nathan J. Weyand, Athens, OH (US)

(21) Appl. No.: 16/516,568

(22) Filed: Jul. 19, 2019

Related U.S. Application Data
(60) Provisional application No. 62/701,038, filed on Jul. 20, 2018.

(51) Int. Cl. A61K 49/00; A01K 67/027

(52) U.S. Cl. CPC A61K 49/0008; A01K 67/027; A01K 2207/12; A01K 2267/0337; A01K 2227/105

(57) ABSTRACT

Provided herein is an animal model system and uses thereof. In particular, provided herein is an animal model for Neisseria infection and use of such a model in research and screening applications.

### CROSS-REFERENCE TO RELATED APPLICATIONS

This application claims priority to and the benefit of U.S. Provisional Application No. 62/701,038, filed Jul. 20, 2018, which is hereby incorporated by reference in its entirety.

### FIELD OF THE INVENTION

Provided herein is an animal model system and uses thereof. In particular, provided herein is an animal model for Neisseria infection and use of such a model in research and screening applications.

### BACKGROUND

Neisseria gonorrhoeae (Ngo) and Neisseria meningitidis (Nme) are pathogens that cause high impact diseases in humans. Ngo infects the urinary tract and oropharynx of males and females. Ngo causes over 160 million new infections each year, worldwide. There is currently no vaccine against Ngo. Ngo has developed resistance to all antibiotics used for its treatment, leading the NIH, CDC and WHO to place Ngo on their list of “superbugs”. NIH has announced initiatives to accelerate the development of novel antimicrobials and identify new targets for antimicrobials and antibiotics against these superbugs.

Nme colonizes the upper respiratory tract, entering the bloodstream to cause septicemia and crossing the blood-brain barrier to cause meningitis. Crowded living conditions and large migrations encourage the spread of Nme, and are the main cause of epidemics and micro-epidemics around the world. As the CDC does not require the reporting of Nme infections, there is no accurate information on their incidence. Vaccines have significantly reduced the incidence of meningococcal disease in developed countries. However, they do not cover all Nme serogroups and are unaffordable in poor countries. Nme continues to cause occasional epidemics in parts of Africa and the Middle East.

Ngo and Nme cause disease only in humans. They do not even colonize a rhesus macaque, an animal that is genetically closest to man. For this reason, animal models for Ngo and Nme do not recapitulate infection in humans. The absence of a valid animal model is the biggest barrier to understanding how Ngo and Nme cause infection.

Animal models suitable for screening vaccines and therapeutics against Ngo and Nme are needed.

### SUMMARY OF THE INVENTION

Commensals are important for the proper functioning of multicellular organisms. How a commensal establishes persistent colonization of its host is little understood. Studies of this aspect of microbe-host interactions are impeded by the absence of an animal model.

Accordingly, provided herein is a natural small animal model for identifying host and commensal determinants of colonization and of the elusive process of persistence. In some embodiments, the system couples a commensal bacterium of wild mice, Neisseria musculi, with the laboratory mouse. The pairing of a mouse commensal with its natural host circumvents issues of host restriction. Studies are performed in a natural setting: no antibiotics, hormones, invasive procedures or genetic manipulation of the host is required. A single dose of N. musculi, administered orally, leads to long-term colonization of the oral cavity and gut. Susceptibility to colonization is determined by host genetics and innate immunity. On the part of N. musculi, colonization requires the Type IV pilus. Reagents and powerful tools are readily available for manipulating the lab mouse, allowing easy dissection of host determinants controlling colonization resistance. N. musculi is genetically related to human-dwelling commensal and pathogenic Neisseria and encodes host interaction factors and vaccine antigens of pathogenic Neisseria. The systems and methods described herein provide a natural approach for studying these commonly held factors and antigens in Neisseria-host interactions, and screening therapeutics and vaccines against pathogenic Neisseria.

For example, in some embodiments, provided herein is a system, comprising: a) a laboratory mouse; and b) a strain of Neisseria musculi (Nmus). The present disclosure is not limited to particular strains of Nmus or mice. In some embodiments, the Nmus is Nmus AP2365. In some embodiments, the mouse is a CAST/EiJ or A/J mouse. In some embodiments, the mouse has a S312P polymorphism in the TLR4 gene. In some embodiments, the oral cavity and gut of the mouse is colonized by Nmus.

Further embodiments provide a test agent. The present disclosure is not limited to particular test agents. Examples include, but are not limited to, a vaccine composition or an anti-bacterial compound. In some embodiments, the anti-bacterial compound targets a Ngo or Nme marker (e.g., PilE or PilT). In some embodiments, the anti-bacterial compound is a small molecule. In some embodiments, candidate therapeutics alter IL-6 levels in a subject or mouse (e.g., increase IL-6 levels).

Additional embodiments provide a method of screening a treatment or prevention of bacterial infection, comprising: a) contacting the system described herein with a candidate agent for treatment or prevention of a bacterial infection; and b) determining the ability of the agent to treat or prevent the bacterial infection. In some embodiments, the bacterial infection is infection by Neisseria gonorrhoeae (Ngo) or Neisseria meningitidis (Nme). In some embodiments, the method comprises colonized the gut of the mouse with Nmus before or after contacting with the test agent.

Additional embodiments are described herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A-C. Nmus colonizes the oral cavity (A) and gut (B) of CAST mice, and different sections of the gastrointestinal tract (C). Samples in (A) and (B) are from the same experiment; each mouse is assigned a unique color. Samples in (C) are taken from 3-month colonized CAST mice from a different experiment. CFU: colony forming units; LOD: level of detection.

FIG. 2. MyD88^{-/-} mice have higher N. musculi burdens than parental B6 mice. N. musculi colony forming units (CFU) in oral swabs taken from B6 (black bars) and MyD88⁻/− (white bars) mice. n=9-10 mice/group.

FIG. 3A-B. N. musculi ΔpilE is defective in colonizing the oral cavity (A) and gut (B) of CAST and B6 mice. CFU: colony forming units; WT: wild type N. musculi; AP2365ΔpilE::pilE_{WT}-C10: pilE complemented strain. Each N. musculi strain was assayed in 10 mice. Oral swab and fecal samples from the same mouse are assigned the same color.

FIG. 4. Protocol for inoculation and sampling of N. musculi in mice. CFU: colony forming units; OC: oral cavity; FP: fecal pellet.

FIG. 5A-B. N. musculi persistently colonizes the oral cavity (A) and gut (B) of CAST mice.

FIG. 6. Nmus ΔpilE does not produce pilE mRNA. WT: parental wild type N. musculi; AP2365ΔpilE::pilE_{WT}-C10 and AP2365ΔpilE::pilE_{WT}-C21: pilE complemented strains; rt: reverse transcriptase; gDNA: genomic DNA control.

FIG. 7. OD₆₀₀ of cultures of N. musculi WT, ΔpilE and complemented strain DpilE::pilE_{wt}-C10. Values are the average of 4 independent experiments.

FIG. 8A-D. Colonization with Neisseria musculi results in antibody production. Representative serum from naïve B6 (solid black), colonized B6 (solid black line), B6 mice who failed colonization (dashed black line) colonized MyD88^{-/-} (solid blue line), and colonized CAST (solid red line) was diluted 1:100 and incubated with Nmus AP2365. Secondary antibodies against anti-mouse IgM (A) and IgG (B) were used to determine antibody subclass binding to Nmus. Mean fluorescence intensity (MFI) of IgM (C) and IgG (D). n=4-8 mice/group.

FIG. 9. CAST mice have a blunted response to E. coli LPS stimulation. Splenocytes from B6 (black circle), CAST (red triangles), and MyD88^{-/-} (blue squares) mice were stimulated for 18 hours with increasing concentrations of E. coli O111:B5 LPS. Supernatants were analyzed by cytometric bead array.

FIG. 10. UV-Killed Nmus fails to control host IL-6 production. BMDM cell lines derived from WT, TLR4^{-/-} and MyD88^{-/-} mice were stimulated for 18 hours with 100 ng/ml of E. coli O111:B5 LPS (black bars), Live Nmus MOI 5 (blue bars), or UV-Killed Nmus MOI 5 (green bars).

FIG. 11. CAST mice have a blunted response to Nmus stimulation. Splenocytes from B6 (left axis) or CAST (right axis) mice were stimulated for 18 hours with E. coli O111:B4 LPS (red bars), Live Nmus MOI 5 (blue bars), or Boiled Nmus MOI 5 (green bars).

FIG. 12. IL-6^{-/-} mice are susceptible to Nmus colonization. Colony forming units (CFU) of Nmus recovered from the oral cavity of B6 (A) or IL-6^{-/-} (B) mice.

FIG. 13. CAST×TLR4^{-/-} mice are resistant to Nmus colonization. Colony forming units (CFU) of Nmus recovered from the oral cavity (A) and fecal pellets (B) of B6 (black circles), CAST (red triangles), CAST×TLR4^{-/-} F1 (blue squares), and TLR4^{-/-} (green triangles) mice. Data points indicate individual mice.

FIG. 14. CAST mice have similar cytokine production levels compared to B6 after ODN stimulation Splenocytes from B6 or CAST mice were stimulated for 18 hours with either control scramble ODN 1585 (black bars) or ODN1585 (red bars) at a final concentration of 1 μM.

FIG. 15. Sequence comparison of CAST and B6 TLR4. DNA and protein alignments of SNPs between CAST and B6 TLR4. Serine 312, indicated in yellow, is the site of the proline substitution in CAST. Space filling model of TLR4 (Green), MD-2 (Yellow), and LPS (Red) the S312P is indicated in white.

### DEFINITIONS

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “subject” and “patient” refer to any animal, such as a mammal like a dog, cat, bird, livestock, and preferably a human.

As used herein, the term “pharmaceutical composition” refers to the combination of an active agent with a carrier, inert or active, making the composition especially suitable for therapeutic use.

The terms “pharmaceutically acceptable” or “pharmacologically acceptable”, as used herein, refer to compositions that do not substantially produce adverse reactions, e.g., toxic, allergic, or immunological reactions, when administered to a subject.

As used herein, the term “administration” refers to the act of giving a drug, prodrug, antibody, vaccine, or other agent, or therapeutic treatment to a physiological system (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, by injection (e.g., intravenously, subcutaneously, intratumorally, intraperitoneally, etc.) and the like.

“Coadministration” refers to administration of more than one chemical agent or therapeutic treatment to a physiological system (e.g., a subject or in vivo, in vitro, or ex vivo cells, tissues, and organs). As used herein, administration “in combination with” one or more further therapeutic agents includes simultaneous (concurrent) and consecutive administration in any order. “Coadministration” of therapeutic treatments may be concurrent, or in any temporal order or physical combination.

As used herein, “carriers” include pharmaceutically acceptable carriers, excipients, or stabilizers which are nontoxic to the cell or mammal being exposed thereto at the dosages and concentrations employed. Often the physiologically acceptable carrier is an aqueous pH-buffered solution. Examples of physiologically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants.

The term “antigen” refers to a molecule (e.g., a protein, glycoprotein, lipoprotein, lipid, nucleic acid, or other substance) that is reactive with an antibody specific for a portion of the molecule.

The terms “protein” and “polypeptide” refer to compounds comprising amino acids joined via peptide bonds and are used interchangeably. A “protein” or “polypeptide” encoded by a gene is not limited to the amino acid sequence encoded by the gene, but includes post-translational modifications of the protein.

Where the term “amino acid sequence” is recited herein to refer to an amino acid sequence of a protein molecule, “amino acid sequence” and like terms, such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein. The term “portion” when used in reference to a protein (as in “a portion of a given protein”) refers to fragments of that protein. The fragments may range in size from four amino acid residues to the entire amino sequence minus one amino acid (for example, the range in size includes 4, 5, 6, 7, 8, 9, 10, or 11 . . . amino acids up to the entire amino acid sequence minus one amino acid).

As used herein, a “vaccine” comprises one or more immunogenic antigens intentionally administered to induce acquired immunity in the recipient (e.g., a subject).

### DETAILED DESCRIPTION

Commensals play an important role in the physiology of multicellular organisms. They are required for the homeostasis of many bodily processes, and they participate in gut and immune system development and prevent pathogen colonization. Perturbations in these microbial communities are strongly linked to obesity, inflammatory bowel disease, diabetes and autoimmunity (Campisi L, et al., 2016. Nat Immunol 17:1084-92; Hancock V, et al. 2010. J Med Microbiol 59:392-9; Iwase T, et al., 2010. Nature 465:346-9; Kugelberg E. 2016. Nat Rev Immunol 16:534-5; Liu X, et al., 2016. Sci Rep 6:30594; Ridaura V K, et al., 2013. Science 341:1241214; Sommer F, Backhed F. 2013. Nat Rev Microbiol 11:227-38).

The mechanisms underlying host and commensal determinants of persistent colonization are little understood. The majority of commensals cannot be cultured or manipulated genetically (Browne H P, et al., 2016. Nature 533:543-6; Stewart E J. 2012. J Bacteriol 194:4151-60; Walker A W, et al., 2014. Trends Microbiol 22:267-74). Because of host restriction barriers, few animal models provide a natural setting for probing commensal-host interactions. The Neisseria, a genus of Gram-negative β-Proteobacteria, provides an opportunity to develop a natural small animal model for this purpose.

The Neisseria genus contains a large number of genetically related species (Bennett J S, et al., 2014. The Genus Neisseria. pp 881-900. Berlin Heidelberg: Springer-Verlag). The vast majority of these are commensals of hosts ranging from rodents, canids and bovines to nonhuman primates and man (Aho E L, et al., 1987. Infect Immun 55:1009-13; Caugant D A, et al., 2009. Vaccine 27 Suppl 2:B64-70; Linz B, et al., 2000. Mol Microbiol 36:1049-58; Liu G, et al., 2015. Microbiology 161:1297-312; Weyand N J, et al., 2016. Int J Syst Evol Microbiol 66:3585-93; Weyand N J, et al., 2013. Proc Natl Acad Sci USA 110:3059-64). Neisseria gonorrhoeae (Ngo) and Neisseria meningitidis (Nme) are the only two species that cause disease. These pathogens, which only infect man, also behave like a commensal in that they have a tendency to colonize asymptomatically (Gerbase A, et al., 2000. Sex Transm Dis 15; Gerbase A C, et al. 1998. Sex Transm Infect 74 Suppl 1:S12-6; 23). Commensal Neisseria are little studied, and there are no small animal models for colonization. Several mouse models have been developed for pathogenic Neisseria infection; but due to the strict tropism of Ngo and Nme for humans, they are necessarily heterologous systems that require invasive procedures, antibiotics, hormones, and/or the use of transgenes expressing human proteins (Gorringe A R, et al., 2005. Vaccine 23:2214-7; Jerse A E, et al., 2011. Front Microbiol 2:107; Johansson L, et al., 2003. Science 301:373-5; Johswich K O, et al., 2013. PLoS Pathog 9:e1003509).

Experiments described herein resulted in the development of an animal model of Neisseria infection. The model utilizes a new species of commensal Neisseria, Neisseria musculi (Nmus), isolated from the oral cavity of healthy wild mice (Weyand N J, et al., 2016. Int J Syst Evol Microbiol 66:3585-93). Nmus is easily cultured and manipulated in vitro, and is genetically related to other Neisseria. Experiments demonstrated that that Nmus colonizes the oral cavity and gut of lab mice for at least 1 year without causing disease. Long-term colonization is achieved with a single oral dose. Using this model, it was determined that permissiveness to Nmus colonization is strongly influenced by host genetics and by innate, but not adaptive, immunity. On the part of Nmus, colonization requires at least its Type IV pilus (Tfp). Finally, it was determined that Nmus encodes pathogenic Neisseria host interaction factors and vaccine antigens.

Accordingly, provided herein is an animal model of Neisseria infection. The model finds use in research, and screening applications described herein.

For example, in some embodiments, provided herein is a system, comprising: a) a laboratory mouse; and b) a strain of Neisseria musculi (Nmus). The present disclosure is not limited to particular strains of Nmus or mice. In some embodiments, the Nmus is Nmus AP2365. In some embodiments, the mouse is a CAST/EiJ or A/J mouse. In some embodiments, the oral cavity and gut of the mouse is colonized by Nmus.

The mouse model described herein finds use in research (e.g., research into the mechanism of Ngo and Nme pathogenicity) and screening (e.g., drug or vaccine screening). For example, in some embodiments, the mouse model is colonized with Nmus prior to contacting with a test agent or after contacting with the test agent. The present disclosure is not limited to particular test agents. In some embodiments, the test agent is a candidate therapeutic (e.g., anti-microbial agent or antibiotic). The test agent may be a protein, small molecule, nucleic acid, antibody or other molecule.

In some embodiments, the test agent targets a Neisseria specific marker (e.g., PilE or PilT). In some embodiments, the mouse model is used to screen candidate agents by determining the effect of an agent on the level or presence of colonization by Nmus and/or prevention of colonization by Nmus. In such embodiments, potential anti-bacterial compositions are identified.

In test embodiments, the test agent is a vaccine composition comprising a Neisseria antigen. In some embodiments, candidate vaccines are screening for their ability to prevent colonization by Nmus. For example, in some embodiments, vaccines are administered to the mouse prior to infection with Nmus and the effect of the vaccine on colonization by Nmus is assayed.

In some embodiments, the present invention relates to a pharmaceutical composition comprising an agent for treating or preventing (e.g., a vaccine composition) Ngo or Nme. In some embodiments, such pharmaceutical compositions can be used as a medicament (e.g., for purposes of treating a subject having gonorrhea and/or any condition involving the presence of Ngo or Nme). In some embodiments, the treatment can be ameliorating, curative or prophylactic treatment of gonorrhea and/or any condition involving the presence of Ngo or Nme.

In some embodiments, the composition is a gel (e.g., formulated for delivery to a mucosal surface). In some embodiments, the gel coats a product for use in treating or preventing infection by Ngo or Nme (e.g., a condom).

In some embodiments, the composition is formulated for delivery to the oropharynx (e.g., as a toothpaste or mouthwash).

Such compositions may be administered using one or more of the following routes of administration. Indeed, routes of administration can broadly be divided into: Topical: local effect, substance is applied directly where its action is desired; Enteral: desired effect is systemic (non-local), substance is given via the digestive tract; Parenteral: desired effect is systemic, substance is given by routes other than the digestive tract.

Topical administration includes Epicutaneous (application onto the skin), Inhalational, Enema, Eye drops (onto the conjunctiva), Ear drops, Intranasal route (into the nose), and Vaginal.

Enteral administration is any form of administration that involves any part of the gastrointestinal tract and includes by mouth (peroral), by gastric feeding tube, duodenal feeding tube, or gastrostomy, and/or rectally.

Parenteral by injection or infusion include Intravenous (into a vein), Intraarterial (into an artery), Intramuscular (into a muscle), Intracerebral (into the cerebrum) direct injection into the brain, Intracerebroventricular (into the cerebral ventricles) administration into the ventricular system of the brain, Intracardiac (into the heart), Subcutaneous (under the skin), Intraosseous infusion (into the bone marrow) is, in effect, an indirect intravenous access because the bone marrow drains directly into the venous system, Intradermal, (into the skin itself), Intrathecal (into the spinal canal), Intraperitoneal, (infusion or injection into the peritoneum), Intravesical infusion is into the urinary bladder, and Intracavernosal injection is into the base of the penis. Other parenteral administration modes include Transdermal (diffusion through the intact skin), Transmucosal (diffusion through a mucous membrane), e.g. insufflation, sublingual, i.e. under the tongue, vaginal suppositories, Inhalational, Intracisternal: given between the first and second cervical vertebrae, Other epidural (synonym: peridural) (injection or infusion into the epidural space), and Intravitreal, through the eye.

Peroral intake may be in the form of Tablets, Capsules, Mixtures, Liquid, and Powder.

Injections may be either systemic or local injections.

Other administration modes of the present invention include Jet-infusion (micro-drops, micro-spheres, micro-beads) through skin, Drinking solution, suspension or gel, Inhalation, Nose-drops, Eye-drops, Ear-drops, Skin application as ointment, gel, lotion, cream or through a patch, Vaginal application as ointment (e.g., via condum, spermacide ointment, etc.), gel, crème or washing, Gastro-Intestinal flushing, and Rectal washings or by use of suppositories.

Administration can be performed as a single administration such as single intake, injection, application, washing; multiple administrations such as multiple intakes, injections, applications, washings; on a single day basis or over prolonged time as days, month, years.

A dose or dosage of the composition according to the present invention may be given as a single dose or in divided doses. A single dose occurs only once, with the drug administered either as a bolus or by continuous infusion. Alternatively, the dose may be divided into multiple doses and given recurrently, such as twice (two times), for example three times, such as four times, for example five times, such as six times, for example seven times, such as eight times, for example nine times, such as ten divided doses. Furthermore, the dose may be given repeatedly, i.e. more than once, such as twice (two times), for example three times, such as four times, for example five times, such as six times, for example seven times, such as eight times, for example nine times, such as ten times a day. Alternatively, the dose may be in sustained release form. A bolus is in theory regarded as given immediately, and should be administered in less than 5 minutes.

It follows that the composition according to the present invention may be given once or more daily, or alternatively may be given with intervals of 1 day, such as 2 days, for example 3 days, such as 4 days, such as 5 days, for example 6 days, such as 7 days (1 week), for example 8 days, such as 9 days, such as 10 days, for example 11 days, such as 12 days, for example 13 days, such as 14 days (2 weeks), such as 3 weeks, for example 4 weeks, such as 5 weeks, for example 6 weeks, such as 7 weeks, such as 8 weeks, for example 12 weeks.

The composition according to the present invention is given in an effective amount to an individual in need thereof. The amount of composition according to the present invention in one preferred embodiment is in the range of from about 0.01 milligram per kg body weight per dose to about 1000 milligram per kg body weight per dose. In some embodiments, the effective amount is the amount necessary for the composition to induce Ngo growth inhibition and/or killing of Ngo.

The composition according to the present invention can be co-administered to an individual in need thereof in combination with one or more drugs such as one or more drugs with antibacterial effect. The one or more antibiotics can be selected from the group consisting of Amikacin disulfate salt, Amikacin hydrate, Anisomycin from Streptomyces griseolus, Apramycin sulfate salt, Azithromycin, Blasticidine S hydrochloride, Brefeldin A, Brefeldin A from Penicillium brefeldianum, Butirosin sulfate salt, Butirosin A from Bacillus vitellinus, Chloramphenicol, Chloramphenicol base, Chloramphenicol succinate sodium salt, Chlortetracycline hydrochloride, Chlortetracycline hydrochloride from Streptomyces aureofaciens, Clindamycin 2-phosphate, Clindamycin hydrochloride, Clotrimazole, Cycloheximide from microbial, Demeclocycline hydrochloride, Dibekacin sulfate salt, Dihydrostreptomycin sesquisulfate, Dihydrostreptomycin solution, Doxycycline hyclate, Duramycin from Streptoverticillium cinnamoneus, Emetine dihydrochloride hydrate), Erythromycin, Erythromycin USP, Erythromycin powder, Erythromycin, Temephos, Erythromycin estolate, Erythromycin ethyl succinate, Erythromycin standard solution, Erythromycin stearate, Fusidic acid sodium salt, G 418 disulfate salt, G 418 disulfate salt powder, G 418 disulfate salt solution liquid, Gentamicin solution liquid, Gentamicin solution, Gentamicin sulfate Micromonospora purpurea, Gentamicin sulfate salt, Gentamicin sulfate salt powder USP, Gentamicin-Glutamine solution liquid, Helvolic acid from Cephalosporium caerulens, Hygromycin B Streptomyces hygroscopicus, Hygromycin B Streptomyces hygroscopicus powder, Hygromycin B solution Streptomyces hygroscopicus, Josamycin, Josamycin solution, Kanamycin B sulfate salt, Kanamycin disulfate salt from Streptomyces kanamyceticus, Kanamycin monosulfate from Streptomyces kanamyceticus, Kanamycin monosulfate from Streptomyces kanamyceticus powder USP, Kanamycin solution from Streptomyces kanamyceticus, Kirromycin from Streptomyces collinus, Lincomycin hydrochloride, Lincomycin standard solution, Meclocycline sulfosalicylate salt, Mepartricin, Midecamycin from Streptomyces mycarofaciens, Minocycline hydrochloride crystalline, Neomycin solution, Neomycin trisulfate salt hydrate, Neomycin trisulfate salt hydrate powder, Neomycin trisulfate salt hydrate USP powder, Netilmicin sulfate salt, Nitrofurantoin crystalline, Nourseothricin sulfate, Oleandomycin phosphate salt, Oleandomycin triacetate, Oxytetracycline dihydrate, Oxytetracycline hemicalcium salt, Oxytetracycline hydrochloride, Paromomycin sulfate salt, Puromycin dihydrochloride from Streptomyces alboniger, Rapamycin from Streptomyces hygroscopicus, Ribostamycin sulfate salt, Rifampicin, Rifamycin SV sodium salt, Rosamicin Micromonospora rosaria, Sisomicin sulfate salt, Spectinomycin dihydrochloride hydrate, Spectinomycin dihydrochloride hydrate powder, Spectinomycin dihydrochloride pentahydrate, Spiramycin, Spiramycin from Streptomyces sp., Spiramycin solution, Streptomycin solution, Streptomycin sulfate salt, Streptomycin sulfate salt powder, Tetracycline, Tetracycline hydrochloride, Tetracycline hydrochloride USP, Tetracycline hydrochloride powder, Thiamphenicol, Thiostrepton from Streptomyces azureus, Tobramycin, Tobramycin sulfate salt, Tunicamycin A₁ homolog, Tunicamycin C₂ homolog, Tunicamycin Streptomyces sp., Tylosin solution, Tylosin tartrate, Viomycin sulfate salt, Virginiamycin Mi, (S)-(+)-Camptothecin, 10-Deacetylbaccatin III from Taxus baccata, 5-Azacytidine, 7-Aminoactinomycin D, 8-Quinolinol crystalline, 8-Quinolinol hemisulfate salt crystalline, 9-Dihydro-13-acetylbaccatin III from Taxus canadensis, Aclarubicin, Aclarubicin hydrochloride, Actinomycin D from Streptomyces sp., Actinomycin I from Streptomyces antibioticus, Actinomycin V from Streptomyces antibioticus, Aphidicolin Nigrospora sphaerica, Bafilomycin A1 from Streptomyces griseus, Bleomycin sulfate from Streptomyces verticillus, Capreomycin sulfate from Streptomyces capreolus, Chromomycin A₃ Streptomyces griseus, Cinoxacin, Ciprofloxacin BioChemika, cis-Diammineplatinum(II) dichloride, Coumermycin A1, Cytochalasin B Helminthosporium dematioideum, Cytochalasin D Zygosporium mansonii, Dacarbazine, Daunorubicin hydrochloride, Daunorubicin hydrochloride USP, Distamycin A hydrochloride from Streptomyces distallicus, Doxorubicin hydrochloride, Echinomycin, Echinomycin BioChemika, Enrofloxacin BioChemika, Etoposide, Etoposide solid, Flumequine, Formycin, Fumagillin from Aspergillus fumigatus, Ganciclovir, Gliotoxin from Gliocladium fimbriatum, Lomefloxacin hydrochloride, Metronidazole purum, Mithramycin A from Streptomyces plicatus, Mitomycin C Streptomyces caespitosus, Nalidixic acid, Nalidixic acid sodium salt, Nalidixic acid sodium salt powder, Netropsin dihydrochloride hydrate, Nitrofurantoin, Nogalamycin from Streptomyces nogalater, Nonactin from Streptomyces tsusimaensis, Novobiocin sodium salt, Ofloxacin, Oxolinic acid, Paclitaxel from Taxus yannanensis, Paclitaxel from Taxus brevifolia, Phenazine methosulfate, Phleomycin Streptomyces verticillus, Pipemidic acid, Rebeccamycin from Saccharothrix aerocolonigenes, Sinefungin, Streptonigrin from Streptomyces flocculus, Streptozocin, Succinylsulfathiazole, Sulfadiazine, Sulfadimethoxine, Sulfaguanidine purum, Sulfamethazine, Sulfamonomethoxine, Sulfanilamide, Sulfaquinoxaline sodium salt, Sulfasalazine, Sulfathiazole sodium salt, Trimethoprim, Trimethoprim lactate salt, Tubercidin from Streptomyces tubercidicus, 5-Azacytidine, Cordycepin, Formycin A, (+)-6-Aminopenicillanic acid, 7-Aminodesacetoxycephalosporanic acid, Amoxicillin, Ampicillin, Ampicillin sodium salt, Ampicillin trihydrate, Ampicillin trihydrate USP, Azlocillin sodium salt, Bacitracin Bacillus licheniformis, Bacitracin zinc salt Bacillus licheniformis, Carbenicillin disodium salt, Cefaclor, Cefamandole lithium salt, Cefamandole nafate, Cefamandole sodium salt, Cefazolin sodium salt, Cefinetazole sodium salt, Cefoperazone sodium salt, Cefotaxime sodium salt, Cefsulodin sodium salt, Cefsulodin sodium salt hydrate, Ceftriaxone sodium salt, Cephalexin hydrate, Cephalosporin C zinc salt, Cephalothin sodium salt, Cephapirin sodium salt, Cephradine, Cloxacillin sodium salt, Cloxacillin sodium salt monohydrate, D-~( )-Penicillamine hydrochloride, D-Cycloserine microbial, D-Cycloserine powder, Dicloxacillin sodium salt monohydrate, D-Penicillamine, Econazole nitrate salt, Ethambutol dihydrochloride, Lysostaphin from Staphylococcus staphylolyticus, Moxalactam sodium salt, Nafcillin sodium salt monohydrate, Nikkomycin, Nikkomycin Z Streptomyces tendae, Nitrofurantoin crystalline, Oxacillin sodium salt, Penicillic acid powder, Penicillin G potassium salt, Penicillin G potassium salt powder, Penicillin G potassium salt, Penicillin G sodium salt hydrate powder, Penicillin G sodium salt powder, Penicillin G sodium salt, Phenethicillin potassium salt, Phenoxymethylpenicillinic acid potassium salt, Phosphomycin disodium salt, Pipemidic acid, Piperacillin sodium salt, Ristomycin monosulfate, Vancomycin hydrochloride from Streptomyces orientalis, 2-Mercaptopyridine N-oxide sodium salt, 4-Bromocalcimycin A23187 BioChemika, Alamethicin Trichoderma viride, Amphotericin B Streptomyces sp., Amphotericin B preparation, Calcimycin A23187, Calcimycin A23187 hemi(calcium-magnesium) salt, Calcimycin A23187 hemicalcium salt, Calcimycin A23187 hemimagnesium salt, Chlorhexidine diacetate salt monohydrate, Chlorhexidine diacetate salt hydrate, Chlorhexidine digluconate, Clotrimazole, Colistin sodium methanesulfonate, Colistin sodium methanesulfonate from Bacillus colistinus, Colistin sulfate salt, Econazole nitrate salt, Hydrocortisone 21-acetate, Filipin complex Streptomyces filipinensis Gliotoxin from Gliocladium fimbriatum, Gramicidin A from Bacillus brevis, Gramicidin C from Bacillus brevis, Gramicidin from Bacillus aneurinolyticus (Bacillus brevis), lonomycin calcium salt Streptomyces conglobatus, Lasalocid A sodium salt, Lonomycin A sodium salt from Streptomyces ribosidificus, Monensin sodium salt, N-(6-Aminohexyl)-5-chloro-1-naphthalenesulfonamide hydrochloride, Narasin from Streptomyces auriofaciens, Nigericin sodium salt from Streptomyces hygroscopicus, Nisin from Streptococcus lactis, Nonactin from Streptomyces sp., Nystatin, Nystatin powder, Phenazine methosulfate, Pimaricin, Pimaricin from Streptomyces chattanoogensis, Polymyxin B solution, Polymyxin B sulfate salt, DL-Penicillamine acetone adduct hydrochloride monohydrate, Polymyxin B sulfate salt powder USP, Praziquantel, Salinomycin from Streptomyces albus, Salinomycin from Streptomyces albus, Surfactin from Bacillus subtilis, Valinomycin, (+)-Usnic acid from Usnea dasypoga, (±)-Miconazole nitrate salt, (S)-(+)-Camptothecin, 1-Deoxymannojirimycin hydrochloride, 1-Deoxynojirimycin hydrochloride, 2-Heptyl-4-hydroxyquinoline N-oxide, Cordycepin, 1,10-Phenanthroline hydrochloride monohydrate puriss., 6-Diazo-5-oxo-L-norleucine, 8-Quinolinol crystalline, 8-Quinolinol hemisulfate salt, Antimycin A from Streptomyces sp., Antimycin A₁, Antimycin A₂, Antimycin A₃, Antipain, Ascomycin, Azaserine, Bafilomycin A1 from Streptomyces griseus, Bafilomycin B1 from Streptomyces species, Cerulenin BioChemika, Chloroquine diphosphate salt, Cinoxacin, Ciprofloxacin, Mevastatin BioChemika, Concanamycin A, Concanamycin A Streptomyces sp, Concanamycin C from Streptomyces species, Coumermycin A1, Cyclosporin A from Tolypocladium inflatum, Cyclosporin A, Econazole nitrate salt, Enrofloxacin, Etoposide, Flumequine, Formycin A, Furazolidone, Fusaric acid from Gibberella fujikuroi, Geldanamycin from Streptomyces hygroscopicus, Gliotoxin from Gliocladium fimbriatum, Gramicidin A from Bacillus brevis, Gramicidin C from Bacillus brevis, Gramicidin from Bacillus aneurinolyticus (Bacillus brevis), Gramicidin from Bacillus brevis, Herbimycin A from Streptomyces hygroscopicus, Indomethacin, Irgasan, Lomefloxacin hydrochloride, Mycophenolic acid powder, Myxothiazol BioChemika, N-(6-Aminohexyl)-5-chloro-1-naphthalenesulfonamide hydrochloride, Nalidixic acid, Netropsin dihydrochloride hydrate, Niclosamide, Nikkomycin BioChemika, Nikkomycin Z Streptomyces tendae, N-Methyl-1-deoxynojirimycin, Nogalamycin from Streptomyces nogalater, Nonactin 80% from Streptomyces tsusimaensis, Nonactin from Streptomyces sp., Novobiocin sodium salt, Ofloxacin, Oleandomycin triacetate, Oligomycin Streptomyces diastatochromogenes, Oligomycin A, Oligomycin B, Oligomycin C, Oligomycin Streptomyces diastatochromogenes, Oxolinic acid, Piericidin A from Streptomyces mobaraensis, Pipemidic acid, Radicicol from Diheterospora chlamydosporia solid, Rapamycin from Streptomyces hygroscopicus, Rebeccamycin from Saccharothrix aerocolonigenes, Sinefungin, Staurosporine Streptomyces sp., Stigmatellin, Succinylsulfathiazole, Sulfadiazine, Sulfadimethoxine, Sulfaguanidine purum, Sulfamethazine, Sulfamonomethoxine, Sulfanilamide, Sulfaquinoxaline sodium salt, Sulfasalazine, Sulfathiazole sodium salt, Triacsin C from Streptomyces sp., Trimethoprim, Trimethoprim lactate salt, Vineomycin A₁ from Streptomyces albogriseolus subsp., Tectorigenin, and Paracelsin Trichoderma reesei.

In some embodiments, vaccine compositions comprise one or more different agents in addition to the antigen. These agents or cofactors include, but are not limited to, adjuvants, surfactants, additives, buffers, solubilizers, chelators, oils, salts, therapeutic agents, drugs, bioactive agents, antibacterials, and antimicrobial agents (e.g., antibiotics, antivirals, etc.). In some embodiments, a vaccine composition comprises an agent or co-factor that enhances the ability of the antigenic unit to induce an immune response (e.g., an adjuvant). In some preferred embodiments, the presence of one or more co-factors or agents reduces the amount of antigenic unit required for induction of an immune response (e.g., a protective immune response (e.g., protective immunization)).

Adjuvants are described in general in Vaccine Design—the Subunit and Adjuvant Approach, edited by Powell and Newman, Plenum Press, New York, 1995, incorporated by reference herein in its entirety for all purposes. The present invention is not limited by the type of adjuvant utilized (e.g., for use in a composition (e.g., a pharmaceutical composition)). For example, in some embodiments, suitable adjuvants include an aluminium salt such as aluminium hydroxide gel (e.g., alum) or aluminium phosphate. In some embodiments, an adjuvant may be a salt of calcium, iron, or zinc, or it may be an insoluble suspension of acylated tyrosine, or acylated sugars, cationically or anionically derivatized polysaccharides, or polyphosphazenes.

Several types of Th1-type immunogens can be used (e.g., as an adjuvant) in compositions and methods of the present invention. These include, but are not limited to, the following. In some embodiments, monophosphoryl lipid A (e.g., in particular, 3-de-O-acylated monophosphoryl lipid A (3D-MPL)), is used. One example is 3D-MPL. It is often supplied as a mixture of 3-de-O-acylated monophosphoryl lipid A with either 4, 5, or 6 acylated chains. In some embodiments, diphosphoryl lipid A and 3-O-deacylated variants thereof are used. Each of these immunogens can be purified and prepared by methods described in GB 2122204B, hereby incorporated by reference in its entirety. Other purified and synthetic lipopolysaccharides have been described (See, e.g., U.S. Pat. No. 6,005,099 and EP 0 729 473; Hilgers et al., 1986, Int. Arch. Allergy. Immunol., 79(4):392-6; Hilgers et al., 1987, Immunology, 60(1):141-6; and EP 0 549 074, each of which is hereby incorporated by reference in its entirety). In some embodiments, 3D-MPL is used in the form of a particulate formulation (e.g., having a small particle size less than 0.2 micrometers in diameter, described in EP 0 689 454, hereby incorporated by reference in its entirety).

In some embodiments, saponins are used as an immunogen (e.g., Th1-type adjuvant) in a composition of the present invention. Saponins are well known adjuvants (See, e.g., Lacaille-Dubois and Wagner (1996) Phytomedicine vol 2 pp 363-386). Examples of saponins include Quil A (derived from the bark of the South American tree Quillaja Saponaria Molina), and fractions thereof (See, e.g., U.S. Pat. No. 5,057,540; Kensil, Crit Rev Ther Drug Carrier Syst, 1996, 12 (1-2):1-55; and EP 0 362 279, each of which is hereby incorporated by reference in its entirety). Also contemplated to be useful in the present invention are the haemolytic saponins QS7, QS17, and QS21 (HPLC purified fractions of Quil A; See, e.g., Kensil et al. (1991). J. Immunology 146, 431-437, U.S. Pat. No. 5,057,540; WO 96/33739; WO 96/11711 and EP 0 362 279, each of which is hereby incorporated by reference in its entirety). Also contemplated to be useful are combinations of QS21 and polysorbate or cyclodextrin (See, e.g., WO 99/10008, hereby incorporated by reference in its entirety).

In some embodiments, an immunogenic oligonucleotide containing unmethylated CpG dinucleotides (“CpG”) is used as an adjuvant. CpG is an abbreviation for cytosine-guanosine dinucleotide motifs present in DNA. CpG is known in the art as being an adjuvant when administered by both systemic and mucosal routes (See, e.g., WO 96/02555, EP 468520, Davis et al., J. Immunol, 1998, 160(2):870-876; McCluskie and Davis, J. Immunol., 1998, 161(9):4463-6; and U.S. Pat. App. No. 20050238660, each of which is hereby incorporated by reference in its entirety). For example, in some embodiments, the immunostimulatory sequence is Purine-Purine-C-G-pyrimidine-pyrimidine; wherein the CG motif is not methylated.

In some embodiments, adjuvants such as Complete Freunds Adjuvant and Incomplete Freunds Adjuvant, cytokines (e.g., interleukins (e.g., IL-2, IFN-γ, IL-4, etc.), macrophage colony stimulating factor, tumor necrosis factor, etc.), detoxified mutants of a bacterial ADP-ribosylating toxin such as a cholera toxin (CT), a pertussis toxin (PT), or an E. coli heat-labile toxin (LT), particularly LT-K63 (where lysine is substituted for the wild-type amino acid at position 63), LT-R72 (where arginine is substituted for the wild-type amino acid at position 72), CT-S109 (where serine is substituted for the wild-type amino acid at position 109), and PT-K9/G129 (where lysine is substituted for the wild-type amino acid at position 9 and glycine substituted at position 129) (see, e.g., WO93/13202 and WO92/19265, each of which is hereby incorporated by reference), and other immunogenic substances (e.g., that enhance the effectiveness of a composition of the present invention) are used with a vaccine composition of the present invention.

Additional examples of adjuvants that find use in the present invention include poly(di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.).

### EXPERIMENTAL

### Example I

### Materials and Methods

Generation of the Rifampicin-Resistant N. musculi Strain.

AP2365, a naturally occurring Rifampicin resistant (RifR) variant of Neisseria musculi type strain, was isolated by plating AP2031 on GCB (Becton Dickinson) agar containing Rifampicin (50 mg/L).

### Mouse Strains.

All inbred mouse strains and Collaborative Cross parental strains were obtained from The Jackson Laboratory (Bar Harbor, Me.). All animal protocols were approved by The University of Arizona IACUC.

### Mouse Inoculation Protocol.

Mice were rested in the University of Arizona mouse facility for two weeks before inoculation. The inoculation protocol is shown in FIG. 4. To determine the presence of Neisseria species in the indigenous flora of the animals, the oral cavities of mice were swabbed; the swabs were suspended in GCB medium base (Becton Dickinson) plus Kellogg's Supplement I and II, and dilutions of the suspensions were plated on GCB agar containing Vancomycin (2 mg/L) and Trimethoprim (3 mg/L). The plates were counted after incubation for 48 h at 37° C., 5% CO₂. Neisseria has never been recovered from mice before inoculation. Fecal pellets of mice were suspended and processed similarly. On the day of inoculation, AP2365 was swabbed from agar plate and resuspended in PBS at an OD₆₀₀ of 2.0. Mice were manually restrained and 50 μl of the bacterial suspension was pipetted into the oral cavity. The oral cavities of the inoculated mice were swabbed weekly or biweekly. Swab suspensions in GCB medium base (Becton Dickinson) were plated on GCB agar containing Rifampicin (40 mg/L), and the plates were incubated for 48 h at 37° C., 5% CO₂.

Verification of Neisseria musculi in Oral Swab Suspensions.

Samples from each colony growing on GCB Rifampicin agar were used for verification of Neisseria musculi as described. Briefly, ITS primers specific to sequences that are highly conserved among Neisseria species were used for colony PCR (Weyand N J, et al. 2016. Int J Syst Evol Microbiol 66:3585-93). The ITS sequences of sample isolates were compared to the type strain AP2031 for species validation, and found to be identical.

Construction of N. musculi ΔpilE and its Complemented Strain.

Table 3 lists the primers used for these constructions. In AP2365 ΔpilE, the pilE open reading frame was replaced with a Kanamycin (Kan) resistance cassette. Primers IM011F and IM012R containing flanking sequences for the pilE gene in Neisseria musculi AP2031T were used to amplify the Kan cassette from plasmid pNBNeiKan (Datsenko K A, Wanner B L. 2000. Proc Natl Acad Sci USA 97:6640-5) (synthesized by Genescript). The amplified DNA was purified and transformed into WT Neisseria musculi AP2031T by spot or liquid transformation as described (Dillard J P. 2011. Curr Protoc in Microbiol), and transformants were selected on GCB agar containing Kellogg's Supplements I and II (Kellogg D S, et al., 1963. J. Bacteriol. 85:1274-9) and Kan (50 mg/L). The ΔpilE::kan locus in AP2031T was transferred to the Rifampicin resistant Neisseria musculi strain AP2365 as follows. Primers NP246F and NP246R2 were used to amplify ΔpilE::kan from AP2031T, and the amplified DNA was cloned into pGEMT (Promega). The recombinant plasmid DNA was introduced into AP2365 by spot transformation. Transformants were selected on GCB containing GCB agar containing Supplements I and II and Kan (50 mg/L). The ΔpilE::kan locus in AP2365 was confirmed by Sanger sequencing of PCR products generated with primers NP246F and NP246R2.

The complemented strains AP2365ΔpilE::pilE_{WT}-C10 and AP2365ΔpilE::pilE_{WT}-C21 were constructed as follows. Primers IM013 and IM014 were used to amplify the Chloramphenicol (Cm) cassette from plasmid pLES94 (Higashi D L, et al., 2011. PLoS One 6:e21373). Primers MR485 and MR486 were used to amplify the WT pilE locus in AP2031T. The Cm PCR product was digested with Pacl and EcoRV (New England Biolabs) and the pilE PCR product was digested with Afe1 and 1 KpnI (New England Biolabs). The two digested DNAs were ligated into similarly digested pUC19 (New England Biolabs) using T4 ligase (New England Biolabs). Primers IM0015 and IM0016 were used to amplify the pilE::cm region in the recombinant plasmid, and the amplified DNA was cloned into pGEMT (Promega). DNA from the resulting plasmid was electroporated into the AP2365 ΔpilE::kan to replace the mutated pilE locus. Transformants were selected and maintained on GCB agar containing Supplements I and II and Chlormaphenicol (2.5 mg/L). The pilE locus in the complemented strains was confirmed by Sanger sequencing of PCR products generated with primers NP246F and NP246R2.

### Transformation Assays.

DNA transformations were performed as described (49). Briefly, recipient strains AP2365, AP2365ΔpilE, and AP2365ΔpilE::pilE_{WT}-C10 were grown for 16 h at 37° C. on GCB agar containing Supplements I and II and the appropriate selective antibiotic(s). Bacterial cells were suspended in GCB broth containing MgSO₄ (5 mM). 30 μL of each suspension, previously diluted to an OD₆₀₀ of 1.5, was added to 0.2 mL of GCB liquid containing MgSO₄ (5 mM) and 1 μg of chromosomal DNA from Neisseria musculi strain AP2093, a naturally occurring isolate whose rpsL contains a point mutation confering resistance to Streptomycin (Weyand N J, et al. 2016. Int J Syst Evol Microbiol 66:3585-93). Following incubation at 37° C. for 20 min, bacteria were added to 2 mL of GCB liquid containing Supplements I and II, and incubated at 37° C., 5% CO₂ for 4 h. Transformants were enumerated by plating cells onto GCB agar containing Supplements I and II and Streptomycin (100 mg/mL), and total input bacteria were enumerated by plating an equal volume on supplemented GCB agar without antibiotics.

RNA Extraction, cDNA Synthesis, and RT-PCR.

Bacterial cells were grown to mid-log phase in GCB broth containing Supplements I and II, and total RNA was extracted using Trizol (Invitrogen) according to manufacturer's instructions. Contaminating DNA was removed using DNA-free (Ambion). The quality and amount of RNA was determined by spectrophotometry (NanoDrop, Therm Scientific). For RT-PCR, 1000 μg of RNA were used to generate the first strand using M-MLV reverse transcriptase (Promega), according to manufacturer's instructions. This was followed by a PCR reaction using GoTaq green master mix (Promega). Nmus pilE was amplified using primers MR489 and MR490. Nmus 16S was amplified using primers MR493 and MR494.

### Growth Curves.

Bacterial cells were grown for 16 h at 37° C., 5% CO₂ on GCB agar containing Supplements I and II and the appropriate selective antibiotics. Cells were scraped from the plates, suspended in supplemented GCB, and diluted to an OD₆₀₀ of 0.05. 2 ml of each bacterial sample was added to 60 mm dishes and incubated at 37° C., 5% CO₂. Bacterial density was measured every two h for 10 h using a Beckman Coulter DU730 spectrophotometer (Brea, Calif.). The cell density at each time point was expressed by subtracting the OD₆₀₀ value at t=0 from the OD₆₀₀ value at that time of collection.

### Adherence Assay.

A static biofilm assay adapted from Merritt (38) was used to measure adherence. Briefly, 2 ml supplemented GCB liquid was added to each well of a 6 well dish (Corning) and 1×10⁷ CFU of N. musculi WT, ΔpilE or the complement strain was introduced into the wells. The plates were incubated at 37° C. 5% CO₂ for 16 h. Each well was gently washed 3 times with 1 ml sterile PBS. Any residual wash buffer was forcibly shaken from the plate to remove all planktonic bacteria. One ml of 0.1% crystal violet was added to each well and the plate was incubated for 30 min at RT. The excess dye was removed, and all wells were washed with 10 ml of PBS. Retained crystal violet was solubilized by the addition of 1 ml of 30% glacial acetic acid, and the OD_{550 nm} was measured on a Beckman Coulter DU730 spectrophotometer (Brea, Calif.). Three fields were imaged per before the initial washes and after crystal violet staining. Results are representative of three independent experiments performed in technical triplicate.

### Blast Searches.

TBlastn searches were conducted using TBLASTN 2.7.1+(3; 4). Protein query sequences from N. meningitidis and N. gonorrhoeae were used to search Nmus strain AP2031's genome sequence (PubMLST ID 29520; (Jolley K A, Maiden M C. 2010. BMC Bioinformatics 11:595)). Many Nme queries used for the analysis were retrieved from the Protegen protective antigen database (Yang B, et al., 2011. Nucleic Acids Res 39:D1073-8). The accession numbers for the commensal human-dwelling Neisseria genome data used for BLAST searches are: Npo (Neisseria polysaccharea ATCC 43768, NZ_ADBE00000000), Nla (Neisseria lactamica 02-06, NC_014752), Nci, (Neisseria cinerea ATCC 14685, NZ_ACDY00000000), Nsu, (Neisseria subflava NJ9703, NZ_ACEO00000000), Nor (Neisseria oxalis CCUG 26878, PubMLST ID 19091), Nmu (Neisseria mucosa ATCC 25996, NZ_ACDX00000000), Nel (Neisseria elongata ATCC 29315, NZ_CP007726), Nba (Neisseria bacilliformis ATCC BAA-1200, NZ_AFAY00000000).

### Example 2

N. musculi Colonizes the Oral Cavity and Gut of Mice in a Mouse Strain Specific Manner.

Nmus was isolated from the oral cavity of a wild mouse, Mus musculus domesticus (Weyand N J, et al., 2016. Int J Syst Evol Microbiol 66:3585-93). Repeated attempts to culture Neisseria from the oral cavity of inbred mice from Jackson Labs and Taconic were unsuccessful. Since inbred lab mice do not harbor Neisseria, this provided an opportunity to test the susceptibility of these animals to Nmus colonization.

The Collaborative Cross (CC) is a new powerful tool in mouse genetics that allows the linkage of alleles with phenotypic traits (Aylor D L, et al., 2011. Genome Res 21:1213-22). Nmus was tested on selected CC founder strains. These strains include 5 conventional, widely used inbred strains, and 3 wild-derived inbred strains from distinct Mus musculus subspecies (TABLE 1). The wild derived strains are CAST, from wild mice trapped in Thailand belonging to a distinct subspecies, Mus musculus castaneous; PWK, which was trapped in the Czech Republic and belonging to subspecies Mus musculus musculus; and WSB/EiJ (WSB), which was trapped in Maryland, USA, and belonging to Mus musculus domesticus. The conventional inbred strains are chimeras with varying degrees of genetic relatedness to CAST, PWK and WSB, although their genetic origin is overwhelmingly Mus musculus domesticus (Aylor et al., supra).

The mouse inoculation protocol is shown in FIG. 4. Prior to inoculation, the presence of Neisseria in these animals was determined by plating oral cavity (OC) and fecal pellet (FP) samples on selective agar. Mice have always been culture-negative. The next day, AP2365, a naturally occurring Rifampicin resistant (Rif) derivative of Nmus, was gently pipetted into the OC of the animals, and Nmus counts in OC and FP were determined weekly for 3 months by plating the samples on selective agar. CAST/EiJ (CAST) and A/J mice were very susceptible to colonization (TABLE 1): the OC and FP of 35/40 (87%) of CAST mice and 26/28 (92%) of A/J mice were continuously culture-positive. C57BL/6J (B6) mice were partially resistant to colonization (12/23; 56%). In contrast, NOD, NZO, PWK, WSB and 129S1 mice were highly resistant.

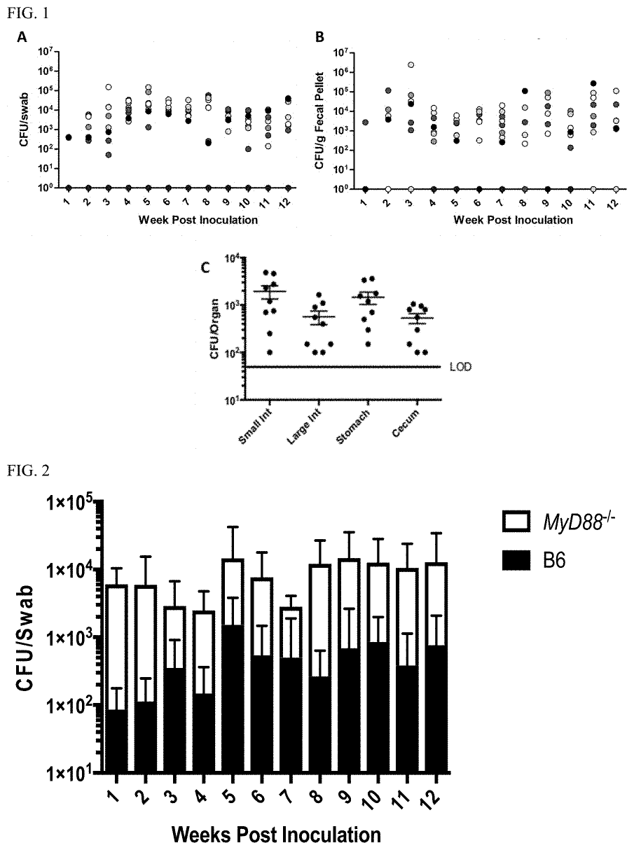

A representative colonization experiment using CAST mice is shown in FIG. 1. Nmus counts in the OC and FP quickly reached a plateau and remained steady thereafter, indicating the commensal had adapted to these niches and replication and turnover reached equilibrium. Generally, when Nmus was cultured from the OC, it was also recovered from the FP, although occasional FP samples were culture-negative (gray and white mice, FIG. 1). These reisolates are Nmus, as judged by multilocus sequence typing of 51 ribosomal genes (rMLST) of 10 OC and 10 FP colonies recovered from CAST mice at 5 weeks post-inoculation.

Two colonized CAST mice were followed long-term. Nmus was continuously recovered from their OC and FP for 52 weeks (FIG. 5). Colonized B6 mice yielded similarly high Nmus counts weekly for 52 weeks.

Throughout the studies, all inoculated and uninoculated mice remained healthy: none lost weight and all maintained healthy coats and normal activity. At necropsy the organs of the 52-week colonized mice reflected those of healthy mice.

Nmus was not cultured from the peripheral blood of 4 CAST and 4 A/J mice 4 hours or 28 days post-inoculation. Although this experiment does not address whether Nmus enters the bloodstream, the result indicate the commensal does not survive at this site.

Taken together, these results demonstrate that the susceptibility of a mouse to Nmus colonization is strongly influenced by its genetic background. In susceptible mouse strains, Nmus easily colonizes their OC and gut, and persists in these niches for lengthy periods without causing disease.

### Example 3

N. musculi Colonizes the Entire Gastrointestinal Tract of Mice.

The location of Nmus in the gastrointestinal tract of 3-month colonized CAST mice was examined. The stomach, small intestine, large intestine and cecum of necropsied animals were flushed with sterile saline to remove luminal content, and the tissues were homogenized and plated on selective agar. Nmus was recovered from all sampled sections of the gut (FIG. 1C). The large numbers of Nmus recovered from tissue-associated gut samples long after inoculation indicates that the commensal was not simply in transit from the OC.

To determine whether Nmus could be horizontally transmitted, 2 colonized CAST mice were cohoused with 3 naïve CAST or B6 mice for 12 weeks. None of the uninoculated mice became colonized. To determine whether the endogenous flora influenced colonization, 4 B6 and 4 CAST mice were cohoused for 12 weeks before inoculation. This did not alter colonization susceptibility of either mouse. Moreover, CAST and B6 mice bred in-house or purchased from The Jackson Labs were always colonized at the same frequency. While only small numbers of mice were used in these co-housing experiments, the evidence indicates that the preexisting flora did not play a significant role in determining colonization susceptibility. To determine whether in vivo passage of Nmus would increase its colonization efficiency, 4 naïve B6 mice were inoculated with Nmus isolated from the OC of a persistently colonized CAST mouse. This in vivo passage did not alter Nmus colonization efficiency. Taken together, these results indicate that neither housing conditions nor the endogenous microbiota are significant roadblocks to Nmus colonization.

### Example 4

Innate Immunity Determines Susceptibility to N. musculi Colonization.

The partial resistance of B6 mice to Nmus colonization (TABLE 1) provided an opportunity to investigate the role of the immune system in determining colonization susceptibility. Nmus was assayed in two strains of immunodeficient B6 mice: B6-MyD88⁻/− mice, which lack the MyD88 adaptor that mediates signaling through many Toll Like Receptors; and B6-Rag-1^{-/-} mice, which lack T and B cells and cannot mount an adaptive immune response (TABLE 2). MyD88^{-/-} mice were exquisitely susceptible to Nmus colonization, unlike the B6 parental strain (MyD88^{-/-} 21/21 mice colonized vs B6 mice 12/23, p<0.001). MyD88^{-/-} mice also had higher Nmus burdens than the parental WT strain (p=0.0026; FIG. 2). In contrast, Rag-1^{-/-} mice were no more susceptible than WT B6 mice. These results indicate that the innate, but not adaptive, immune system is a major determinant of Nmus colonization. The increased numbers of Nmus recovered from MyD88^{-/-} mice, compared to WT B6, indicates that the innate response plays an ongoing role in controlling Nmus numbers.

### Example 5

N. musculi Colonization Requires the Type IV Pilus.

To test the usefulness of the model for studying commensal determinants of colonization, the Type IV pilus (Tfp) was investigated. All Neisseria species have a complete set of Tfp biogenesis genes (Marri P R, et al., 2010. PLoS One 5:e11835; Weyand N J, et al., 2016. Int J Syst Evol Microbiol 66:3585-93; Weyand N J, et al., 2013. Proc Natl Acad Sci USA 110:3059-64). In the case of pathogenic Neisseria, Tfp is implicated to promote colonization, based on experiments using cultured human cells and a limited number of human challenge studies (Hobbs M M, et al., 2011. Front Microbiol 2:123; Kellogg D, et al., 1968. J. Bacteriol. 96:596-605; Kellogg D S, et al., 1963. J. Bacteriol. 85:1274-9; Nassif X, et al., 1993. Mol Microbiol 8:719-25; Swanson J. 1973. J Exp Med 137:571-89). The function of Tfp has never been tested in a natural animal model.

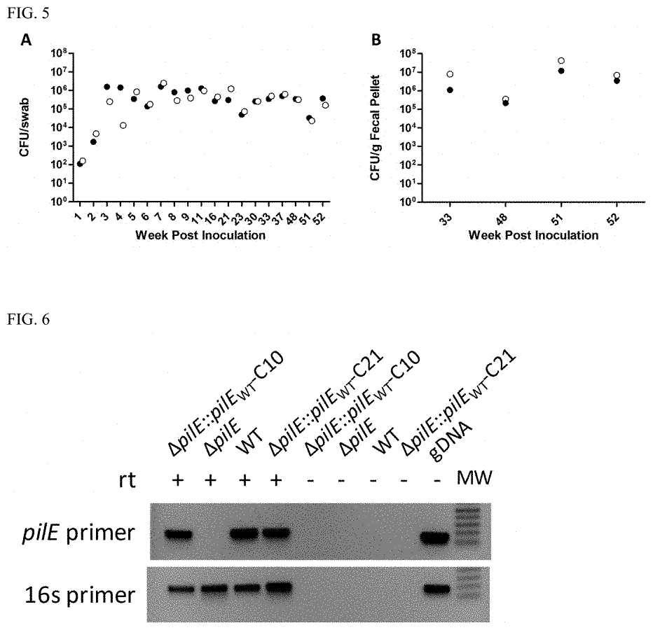

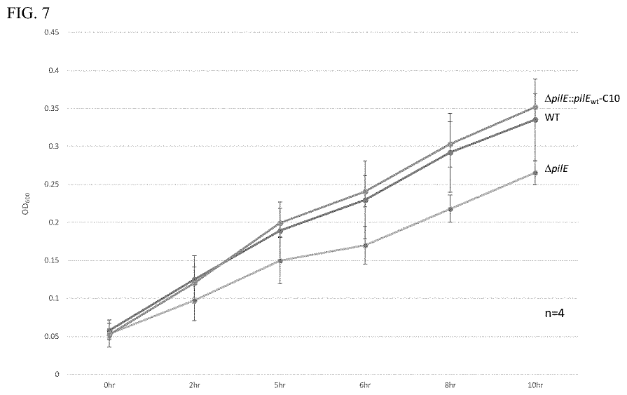

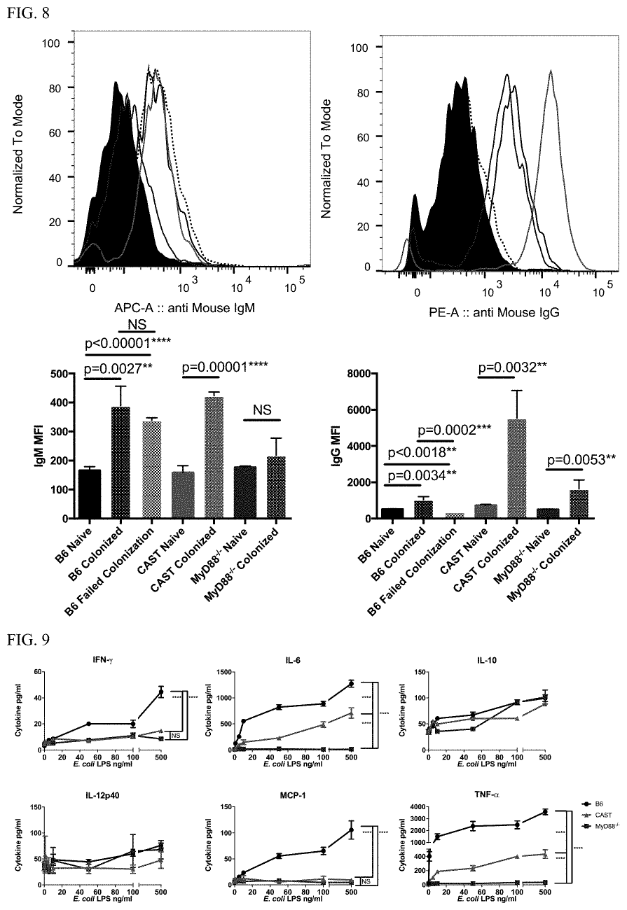

For this experiment, a nonpiliated mutant of Nmus, ΔpilE, was constructed by deleting the gene encoding the Tfp fiber subunit; a complemented strain, ΔpilE::pilE_{WT}-C10, was also constructed. The piliation status of ΔpilE and complement was validated by several methods. Unlike WT and complement, ΔpilE did not produce pilE mRNA, as judged by real time PCR (FIG. 6). Nmus ΔpilE exhibited phenotypes characteristic of nonpiiliated mutants: it was defective in DNA transformation, and attached less well to surfaces (FIG. 7). These results indicate Nmus ΔpilE does not produce the Tfp fiber. Finally, the growth of ΔpilE was examined. WT, ΔpilE and complement grew equally well (FIG. 8). The slightly lower OD₆₀₀ of ΔpilE cultures was not statistically different at any time point; it likely reflects the slight tendency of ΔpilE cells to aggregate in liquid culture.

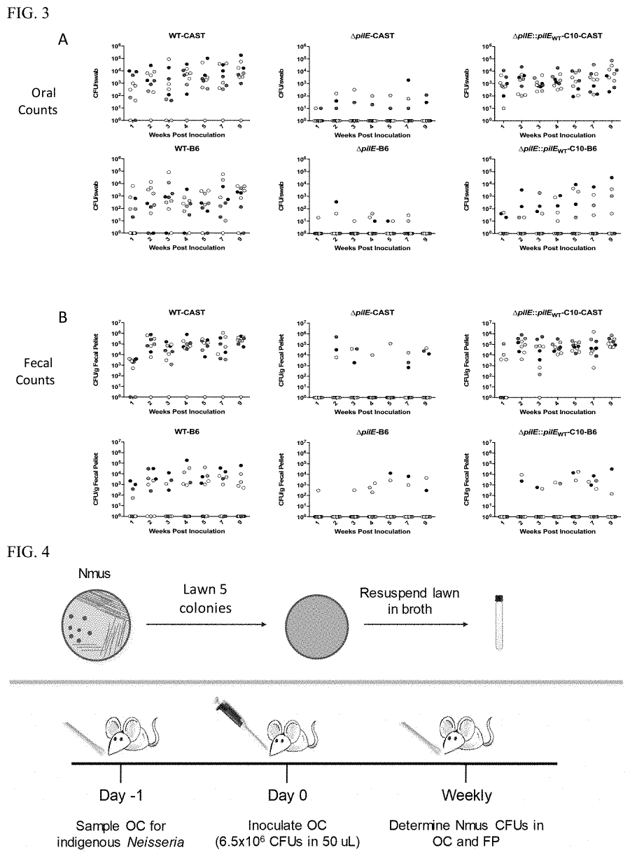

Nmus ΔpwilE was defective in colonizing the OC and gut of CAST and B6 mice, compared to WT and complement (FIG. 3A; p=0.002, WT vs ΔpilE for both CAST and B6). The few OC and FP reisolates are Nmus, as judged by rMLST, and their mutated pilE locus was unaltered. This finding corroborates earlier studies, indicating the Nmus Tfp is important for colonization in vivo.

### Example 6

N. musculi Encodes Host Interaction Factors and Vaccine Candidates of Human-Dwelling Neisseria.

Finally, it was determined whether Nmus could be used to model human-dwelling species of Neisseria. To date, Neisseria colonization studies have focused almost exclusively on the two pathogens, Ngo and Nme. Great efforts have been made to identify host interaction factors, with the goal of identifying vaccine antigens capable of stimulating protective immune responses. Chief among these vaccine development efforts was the use of reverse vaccinology to identify genome-derived Neisseria antigens (GNA) in Nme (Pizza M, et al., 2000. Science 287:1816-20). Currently, similar work is conducted to identify vaccine antigens in Ngo (Jerse A E, et al., 2014. Vaccine 32:1579-87; Zielke R A, et al., 2016. Mol Cell Proteomics 15:2338-55). BLAST searches of the Nmus genome were used to identify putative orthologs of protective antigens from Nme and Ngo. Many homologs of pathogenic Neisseria host interaction factors and candidate vaccine antigens, including GNAs, were found in Nmus and human-dwelling commensal Neisseria. Two GNAs with high identity and query coverage were GNA1220, a membrane protein of unknown function containing a stomatin-like domain; and GNA33, membrane-associated lytic transglycosylase required for cell separation (Adu-Bobie J, et al., 2004. Infect Immun 72:1914-9; Kelly D F, et al., 2005. Adv Exp Med Biol 568:217-23). Nme GNA1946 and Ngo ortholog NGO2139, which are methionine-binding subunits of ABC transporters, both retrieved the same Nmus ortholog with greater than 75% identity and 95% query coverage. GNA1946 and NG02139 (MetQ) induce the production of serum bactericidal antibodies (Pizza M, S et al., 2000. Science 287:1816-20; Semchenko E A, et al., 2017. Infect Immun 85). Nmus also has a homolog for Nme LpdA, a high molecular weight protein, P64k, which is very immunogenic and is used frequently as a carrier protein for weaker immunogens (Gonzalez S, et al., 2000. Scand J Immunol 52:113-6; Lazo L, et al., 2014. Microbiol Immunol 58:219-26).

BLAST searches were conducted using three β-barrel-containing outer membrane proteins as queries: Nme NspA, a factor H ligand, and Ngo adhesins OpaD and OpcA. (The Nme OpcA ortholog is a lectin capable of interacting with vitronectin (Moore J, et al., 2005. J Biol Chem 280:31489-97; Virji M, et al., 1994. Mol Microbiol 14:173-84)). NspA and OpaD retrieved the same Nmus homolog (NspA: 41% identity, 86% query coverage; OpaD: 32% identity, 69% query coverage). OpcA did not have a significant hit.

Capsule transport proteins are crucial for presentation of antigenic capsular polysaccharides in Nme (Harrison O B, et al., 2013. Emerg Infect Dis 19:566-73). Capsule transport genes ctrA-F were found in Nmus and an additional human commensal species, Neisseria oxalis. Taken together, these BLAST results illustrate the utility of the animal model for understanding the in vivo function of host interaction factors and its potential use for assessing the ability vaccine candidate antigens to stimulate immunity or efficacy against carriage.

TABLE 1
Susceptibility of Collaborative Cross founder
strains to colonization by N. musculi

| | | |
|---|---|---|
| Strain | No. colonized/Inoculated (%)^{a} | P value^{b} |

| | | | |
|---|---|---|---|
| CAST/EiJ | 35/40 | (87) | NS^{c} |
| A/J | 26/28 | (92) | |
| C57BL/6J | 12/23 | (52) | <0.006^{c} |
| NOD/LtJ | 0/4 | (0) | <0.0004^{c} |
| NZO/HILtJ | 0/4 | (0) | <0.0004^{c} |
| PWK/PhJ | 0/9 | (0) | <10^{5c} |
| WSB/EiJ | 0/9 | (0) | <10^{5c} |
| 129S1/SvlmJ | 0/4 | (0) | <0.0004^{c} |
| MyD88^{-/-} | 21/21 | (100) | <0.001⁴ |
| RAG-1^{-/-} | 3/14 | (21) | NS^{d} |

^{a}Mice were scored for the presence of N. musculi in the oral cavity and fecal pellet each week for 3 months.
^{b}χ² with Yates correction for small numbers and Bonferroni for multiple pairwise comparisons.
NS, not significant.
^{c}Compared to CAST.
^{d}Compared to WT 8L/6.

TABLE 2
Putative orthologs of protective antigens encoded in the AP2031 genome

| | | | Ortholog | | | | | | | |
|---|---|---|---|---|---|---|---|---|---|---|
| Protein | Query | | N. musculi | | N. polysaccharea | | N. lactamica | | N. cinerea | |
| query | accession no. | Query species | % Id.^{a} | % qc.^{b} | % Id. | % qc. | % Id. | % qc. | % Id. | % qc. |
| LctP | CBA04244 | N. meningitidis | 25 | 95 | 25 | 95 | 98 | 100 | 92 | 100 |
| LpdA | CAA57206 | N. meningitidis | 74 | 100 | 74 | 100 | 85 | 100 | 84 | 100 |
| GNA1030^{c} | NP_274064 | N. meningitidis | 48 | 23 | 29 | 79 | 95 | 88 | 90 | 100 |
| GNA1220 | NP_274245 | N. meningitidis | 81 | 99 | 81 | 99 | 93 | 100 | 93 | 100 |
| GNA1946 | NP_274940 | N. meningitidis | 77 | 95 | 77 | 95 | 88 | 100 | 85 | 100 |
| GNA2091^{c} | NP_275079 | N. meningitidis | 63 | 79 | 63 | 79 | 88 | 100 | 80 | 100 |
| GNA33 | NP_273099 | N. meningitidis | 77 | 85 | 77 | 85 | 91 | 100 | 87 | 100 |
| NadA^{c,d} | NP_274986 | N. meningitidis | 43 | 15 | 43 | 15 | 27 | 30 | 67 | 28 |
| PorA P1^{c,d} | NP_273150 | N. meningitidis | 53 | 97 | 53 | 97 | 86 | 100 | 85 | 100 |
| ExbB | NP_274732 | N. meningitidis | 68 | 99 | 68 | 99 | 96 | 100 | 93 | 99 |
| GNA992 | NP_274028 | N. meningitidis | 48 | 23 | 48 | 23 | 91 | 94 | 39 | 79 |
| GNA2001 | NP_274993 | N. meningitidis | 68 | 65 | 68 | 65 | 65 | 100 | 60 | 100 |
| GNA1870 | NP_274866 | N. meningitidis | 30 | 38 | 30 | 38 | 34 | 61 | 91 | 100 |
| (fHbp)c,d | | | | | | | | | | |
| NspA | NP_273705 | N. meningitidis | 42 | 86 | 42 | 86 | 84 | 87 | 48 | 86 |
| TBP2 | CAA55541 | N. meningitidis | 25 | 13 | 25 | 13 | 74 | 100 | 35 | 98 |
| TbpA | AAF81744 | N. meningitidis | 30 | 54 | 30 | 54 | 94 | 100 | 75 | 100 |
| GNA2132 | NP_275117 | N. meningitidis | ND^{e} | ND | ND | ND | 75 | 100 | 40 | 40 |
| (NHBA)^{c,b} | | | | | | | | | | |
| GNA1162 | NP_274189 | N. meningitidis | 33 | 40 | 33 | 40 | 95 | 100 | 88 | 100 |
| PilC1 | YP_207232 | N. gonorrhoeae | 36 | 68 | 36 | 68 | 46 | 100 | 40 | 89 |
| PilQ | YP_207267 | N. gonorrhoeae | 56 | 100 | 56 | 100 | 81 | 100 | 77 | 100 |
| AniA | YP_208345 | N. gonorrhoeae | 79 | 79 | 79 | 79 | 93 | 79 | 92 | 79 |
| OpaD | YP_208563 | N. gonorrhoeae | 32 | 69 | 32 | 69 | 66 | 100 | 29 | 86 |
| OpcA | CAB45007 | N. gonorrhoeae | 28 | 16 | 28 | 16 | 40 | 64 | 41 | 83 |
| LptD | YP_208748 | N. gonorrhoeae | 60 | 98 | 60 | 98 | 91 | 98 | 81 | 100 |
| BamA | YP_208831 | N. gonorrhoeae | 75 | 100 | 75 | 100 | 90 | 100 | 94 | 100 |
| TamA | YP_208979 | N. gonorrhoeae | 68 | 90 | 68 | 90 | 95 | 100 | 83 | 98 |
| NGO2054 | YP_209073 | N. gonorrhoeae | 64 | 76 | 64 | 76 | 94 | 100 | 78 | 100 |
| NGO2139 | YP_209148 | N. gonorrhoeae | 78 | 95 | 78 | 95 | 90 | 100 | 78 | 100 |
| (MetQ) | | | | | | | | | | |

Putative orthologs of protective antigens encoded in the AP2031 genome
Ortholog

| Protein | N. subflava | | N. oralis | | N. mucosa | | N. elongata | | N. bacilliformis | |
|---|---|---|---|---|---|---|---|---|---|---|
| query | % Id. | % qc. | % Id. | % qc. | % Id. | % qc. | % Id. | % qc. | % Id. | % qc. |
| LctP | 92 | 100 | 92 | 100 | 94 | 100 | 82 | 99 | 40 | 42 |
| LpdA | 85 | 100 | 84 | 100 | 84 | 100 | 71 | 100 | 72 | 100 |
| GNA1030^{c} | 74 | 100 | 86 | 100 | 91 | 86 | 65 | 88 | 62 | 100 |
| GNA1220 | 34 | 31 | 82 | 98 | 84 | 100 | 75 | 99 | 78 | 96 |
| GNA1946 | 79 | 97 | 82 | 97 | 82 | 98 | 80 | 96 | 67 | 96 |
| GNA2091^{c} | 86 | 79 | 67 | 99 | 85 | 80 | 63 | 79 | 65 | 79 |
| GNA33 | 78 | 100 | 73 | 99 | 77 | 100 | 74 | 85 | 67 | 94 |
| NadA^{c,d} | ND^{e} | ND^{e} | 38 | 15 | 69 | 46 | 48 | 7 | 35 | 27 |
| PorA P1^{c,d} | 71 | 98 | 59 | 98 | 71 | 100 | 61 | 99 | 56 | 99 |
| ExbB | 73 | 99 | 73 | 99 | 76 | 99 | 58 | 99 | 57 | 98 |
| GNA992 | 62 | 23 | 64 | 14 | 65 | 29 | 63 | 67 | 64 | 18 |
| GNA2001 | 60 | 97 | 56 | 100 | 56 | 97 | 79 | 98 | 76 | 57 |
| GNA1870 | 39 | 83 | 31 | 62 | 38 | 82 | 28 | 89 | 29 | 64 |
| (fHbp)c,d | | | | | | | | | | |
| NspA | 45 | 86 | 29 | 36 | 40 | 43 | 47 | 86 | 45 | 86 |
| TBP2 | 26 | 8 | 28 | 17 | 31 | 12 | 26 | 10 | 28 | 47 |
| TbpA | 26 | 58 | 28 | 61 | 30 | 71 | 31 | 67 | 32 | 54 |
| GNA2132 | 32 | 25 | 34 | 9 | 35 | 51 | 32 | 29 | 28 | 34 |
| (NHBA)^{c,b} | | | | | | | | | | |
| GNA1162 | 48 | 13 | 35 | 14 | 54 | 99 | 48 | 35 | 52 | 10 |
| PilC1 | 40 | 65 | 36 | 67 | 38 | 66 | 24 | 65 | 26 | 31 |
| PilQ | 61 | 100 | 57 | 99 | 58 | 100 | 49 | 97 | 49 | 97 |
| AniA | 87 | 79 | 80 | 79 | 89 | 79 | 80 | 79 | 78 | 79 |
| OpaD | 32 | 85 | 30 | 19 | 23 | 16 | 32 | 89 | 29 | 85 |
| OpcA | 25 | 76 | 20 | 76 | 31 | 15 | 26 | 82 | 23 | 38 |
| LptD | 63 | 98 | 61 | 98 | 61 | 98 | 55 | 100 | 55 | 92 |
| BamA | 82 | 100 | 80 | 100 | 82 | 100 | 71 | 100 | 69 | 100 |
| TamA | 77 | 89 | 74 | 88 | 75 | 88 | 63 | 89 | 62 | 89 |
| NGO2054 | 62 | 100 | 57 | 81 | 60 | 57 | 54 | 100 | 62 | 74 |
| NGO2139 | 82 | 97 | 86 | 97 | 85 | 98 | 82 | 96 | 77 | 82 |
| (MetQ) | | | | | | | | | | |

^{a}%id., percent identity. Boldface indicates >50% identity.
^{b}%qc., percent query coverage. Italics indicate >75% query coverage.
^{c}Component of rMenB-OMV vaccine (Novartis).
^{d}Component of the Bexsero and Trumemba vaccines (GlaxoSmithKline/Novartis and Pfizer).
^{e}ND, significant similarity not detected.

TABLE 3
Orthologs of N. meningitidis capsule synthesis, transport, and translocation
proteins and presence of selected capsule transcripts in N. musculi

| Protein | Query | | Maximum | Query | | |
|---|---|---|---|---|---|---|
| query | accession no. | Query species | Identity (%)^{a} | coverage (%)^{b} | Genome annotation | mRNA^{e} |
| CssA | WP_002233375.1 | N. meningitidis | 72 | 96 | UDP-N-acetylglucosamine-2-epimerase | + |
| CssB | WP_002233374.1 | N. meningitidis | 87 | 99 | UDP-N-acetyl-D-mannosamine dehydrogenase | ND |
| CssC | CCP19843.1 | N. meningitidis | 28 | 13 | Capsule polymerase | ND |
| CtrA | NP_273135 | N. meningitidis | 56 | 93 | Capsule transport complex | + |
| CtrB | NP_273136 | N. meningitidis | 67 | 91 | Capsule transport complex | ND |
| CtrC | NP_273137 | N. meningitidis | 73 | 100 | Capsule transport complex | ND |
| CtrB | NP_273138 | N. meningitidis | 84 | 98 | Capsule transport complex | ND |
| CtrE | NP_273145 | N. meningitidis | 60 | 93 | Capsule translocation | + |
| CtrF | NP_273146 | N. meningitidis | 61 | 99 | Capsule translocation | + |

^{a}Boldface, sequence identity >50%.
^{b}Italics, query coverage >75%.
^{e}+, positive; ND, not determined.

### Example 7

Type IV Pilus Retraction is Essential for Neisseria musculi Persistent Colonization In Vivo.

The Type IV pilus (Tfp) controls many Neisseria-host interactions. In cultured cells, Tfp retraction triggers mechanosensitive host pathways, culminating in the formation of a cytoprotective environment that promotes bacterial survival. Tfp retraction is also important for biofilm formation in vitro. The model described in Examples 1-6 was used to examine the role of Tfp retraction in Nmus colonization and persistence.

### Methods

Nmus mutants deleted of the Tfp retraction motor gene (DpilT) or expressing an attenuated retraction motor (pilT_{L201C}) were constructed and validated for growth and for Tfp functions using DNA transformation, adhesion, invasion, and biofilm formation assays. Wt, DpilT and pilT_{L201C} were inoculated into CAST/EiJ mice, and CFUs in the oral cavity (OC) and fecal pellet (FP) were determined weekly for 16 weeks. CFUs along the alimentary tract, lungs, liver, spleen and kidneys were also determined at Week 16.

### Results

Like N. gonorrhoeae DpilT and pilT_{L201C}, Nmus DpilT is non-transformable and pilT_{L201C} is genetically competent. DpilT and pilT_{L201C} adhered to cultured mouse epithelial cells as well as wt, but had invasion and biofilm formation defects.

Wt Nmus stably colonized the OC and gut of mice, while DpilT did not colonize these sites at any time. As DpilE has an identical defect, this indicates colonization requires not only the Tfp fiber but also the ability of the fiber to retract.

pilT_{L201C} had yet a different phenotype. pilT_{L201C} colonized the mouse OC and gut, but CFUs from these sites varied from week to week. Taking into account its biofilm defect, the cyclic recovery of pilT^{L201C} CFUs is likely caused by the inability of the mutant to maintain a stable niche on the mucosa.

In conclusion, this example demonstrates that niche establishment requires the presence of a retractable Tfp fiber, and niche maintenance requires a fully functional PilT ATPase.

### Example 8

### Materials and Methods

This Example describes methods for Examples 9-15.

### Mice

See Table 4 for the source and origin of mice used. All animal protocols were approved by The University of Arizona IACUC.

### Colonization of Mice

All mice were placed in an SPF, BSL2 room for 2 weeks before inoculation. One week before the inoculation, the oral cavity was swabbed using the BD BBLTM CultureSwab Plus Transport System (Fisher Scientific) for indigenous Neisseria. Pre-inoculation swab suspensions in GCB medium base (Becton Dickinson) were plated on GCB agar containing Vancomycin (2 μg/ml) and Trimethoprim (3 μg/ml), and the plates were incubated for 48 hours at 37° C., 5% CO₂. No Neisseria were recovered from any mouse before inoculation (Ma, M., et al., 2018. A Natural Mouse Model for Neisseria Colonization. Infect Immun 86). On the day of inoculation, AP2365 was swabbed from agar plate and resuspended in PBS at an OD₆₀₀ of 2.0. Mice were manually restrained and 50 μl of the bacterial suspension was pipetted into the oral cavity. The oral cavities of the inoculated mice were swabbed weekly or biweekly. Swab suspensions in GCB medium base (Becton Dickinson) were plated on GCB agar containing Rifampicin (40 ug/ml), and the plates were incubated for 48 hours at 37° C., 5% CO₂. The colonization frequency of Neisseria musculi were calculated from the counting the colony forming units after 48 hours.

### Cellular Stimulation

Spleens were aseptically removed from 6-8-week-old mice. Spleens were ground over 70 μm cell strainers to produce a single cell suspension. Red blood cells were lysed with ACK lysis buffer. Remaining cells were resuspended in cDMEM (10% FBS, 10 mM L-glutamine, 1 mM Sodium Pyruvate). BMDM transformed by J2 virus were a kind gift from Katherine Fitzgerald (University of Massachusetts). Cells were seeded into 96 well plates at 2.5×10⁵ cells/well in 200 μl of cDMEM. For bacterial stimulations Neisseria musculi AP2365 was grown to mid-log in GCB liquid culture. Bacteria were then pelleted and resuspended in sterile PBS. The suspension was adjusted to give an MOI of 5 in 50 μl of PBS. Heat killed cultures were then incubated at 100° C. for 30 minutes. UV irradiated cultures were exposed to direct UV light for 30 minutes. Sterility was confirmed by plating. All boiled and UV inactivated preps contained less than 1 live CFU/50 μl. Ultrapure E. coli LPS was purchased from Sigma and derived from E. coli O111:B4. Nmus LPS was isolated following the hot phenol method (Darveau, R. P., and R. E. Hancock. 1983. J Bacteriol 155: 831-838; Fischer, W., et al., 1983. Eur J Biochem 133: 523-530). Briefly, 500 mg of lyophilized bacterial pellet was solubilized in a 10 mM Tris-Cl buffer, pH 8.0, with 2% sodium dodecyl sulfate (SDS), 4% β-mercaptoethanol, 20 mg/ml proteinase K, and 2 mM MgCl₂ at 65° C. for 1 h with intermittent vortexing and further digested overnight at 37° C. The samples were precipitated overnight at 20° C. with the addition of sodium acetate to a final concentration of 0.1 M and cold ethanol to 75%. LPS was pelleted, and the precipitation was repeated 2 more times to remove residual SDS and peptides. The samples were then suspended in a 10 mM Tris-Cl buffer, pH 7.4, and digested for 4 h at 37° C. with 100 μg/ml DNase and 25 μg/ml RNase. An equal volume of 90% phenol was added, and the sample incubated at 65° C. for 15 min with occasional vortexing. The sample was cooled in ice-water and centrifuged, and the aqueous fraction was collected. The phenol layer was reextracted with an equal volume of endotoxin-free water. The aqueous layers were pooled and dialyzed in a 1-kDa molecular-mass-cutoff dialysis bag with repeated water changes at 4° C. to remove phenol over 48 h. The samples were then frozen on dry ice and lyophilized. Dry samples were washed four times with 2:1 (vol/vol) chloroform-methanol to remove contaminating hydrophilic lipids and reextracted using the Hirschfeld et al. procedure to remove contaminating lipoproteins (Folch, J., et al., 1957. J Biol Chem 226: 497-509). Samples were then lyophilized and stored sealed at room temperature. Cells were stimulated for 18 hours at 37° C. and 5% CO₂. At 18 hours supernatant was harvested and stored at −80° C. until analysis. Cell supernatants were analyzed using BD Mouse Inflammatory Cytometric Bead Array per the manufacturer's instructions.

### Example 9

### Nmus Colonized Mice Produced an Antibody Response

The major impact of MyD88 deficiency in allowing colonization suggested that Nmus might be undetected by the host immune system. As a simple measure of recognition by the adaptive immune system, colonized mice were tested for Nmus-specific antibodies. In order to detect Nmus specific antibodies, a flow cytometry based binding assay that measured antibody in serum that was able to bind Nmus cells in vitro was developed and used to detect binding with a labeled secondary antibody. It was contemplated that colonization of mice in the oral cavity and gastrointestinal tract would not provoke a strong systemic antibody response. Contrary to expectations, colonized CAST mice had Nmus-specific IgM and IgG antibodies (FIGS. 9A, B). Uninoculated CAST and B6 mice had no detectable antibody. This demonstrates that colonization is detected by the adaptive immune response. Since both IgM and IgG are detected, it indicates T cell involvement (Snapper, C. M., et al., 1997. Immunity 6: 217-223).

### Example 10

### Nmus Specific IgG is Only Produced Following Successful Colonization

The partial susceptibility of B6 mice to Nmus presented an opportunity to compare antibody responses in inoculated mice that became colonized to those that did not do so. The same inoculum dose was used for all mice. As in CAST mice, colonized B6 mice produced both IgM and IgG antibodies. B6 mice that were not colonized produced IgM antibodies (FIG. 8C), but no detectable IgG (FIG. 8D), indicating they had been transiently exposed to Nmus (FIG. 8C). These results show that successful colonization is not an immunologically silent event. The mice that were colonized produce a productive T and B cell response, based on the production of IgG which requires CD4 T cell involvement. Moreover, Nmus persisted in both the OC and gut of mice in the presence of a B and T cell response. Although we have not determined if there is robust antibody response in the OC, the presence of an antibody response suggests that a systemic response does not result in clearance of Nmus. Some previous studies have suggested that host antibody response potentiates colonization by bacteria in germ free mice (Shroff, K. E., et al., 1995. Infect Immun 63: 3904-3913).

### Example 11

### CAST Mice Respond Poorly to MyD88 Dependent PAMPs

Both CAST and MyD88 deficient mice are effectively colonized by Nmus. It was contemplated that the lack of MyD88 response in susceptible mice may be the cause of Nmus colonization. The ability of CAST spleen cells to respond to MyD88 dependent PAMPs was investigated. Splenocytes from CAST, B6 and MyD88^{-/-} were stimulated in vitro for 18 hours with increasing concentrations of E. coli O111:B4 LPS (TLR4 dependent) or ODN1585 (TLR9 dependent). Supernatants were tested for proinflammatory cytokines IFN-γ IL-6, IL-10, IL-12p70, MCP-1, and TNF-α. As expected, neither LPS nor ODN1585 induced cytokine production in MyD88^{-/-} splenocytes. IL-12p70 was not detected at any LPS concentration in any of the cultures. B6 splenocytes responded to LPS by producing IFN-γ, IL-6, MCP-1, and TNF-α in a dose-dependent manner (FIG. 9). CAST splenocytes produced no detectable MCP-1. While CAST mice did produce some IFN-γ, TNF-α and IL-6 the amounts were significantly lower than those found in the supernatants of B6 stimulated cells, but higher than MyD88^{-/-} cells. In contrast, there was no difference in the response to the TLR9 ligand ODN1585 (FIG. 14). These data indicate the TLR4, but not the TLR9, response is blunted in CAST mice compared to B6 mice.

### Example 11

CAST Mice have a Unique SNP in the TLR4 Coding Sequence

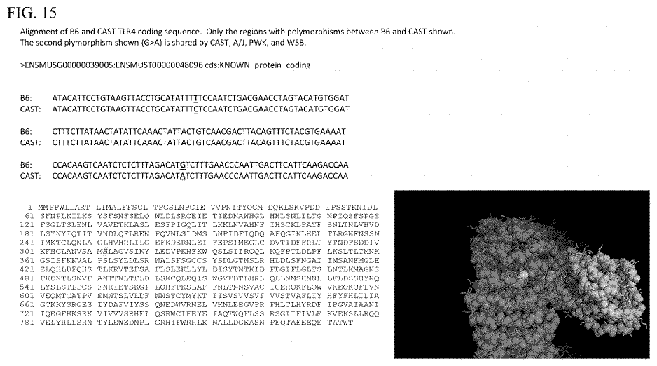

CAST mice are an inbred strain derived from the subspecies Mus. m. castaneus and they differ in many loci from the standard inbred mice which are largely derived from Mus m. musculus (Ideraabdullah, F. Y., et al., 2004. Genome Res 14: 1880-1887; Silver, L. M. 1995. Mouse genetics: concepts and applications. Oxford University Press, New York). If CAST mice differed from standard inbred strains in their LPS receptor complex (TLR4, MD2 or CD14), it may explain increased CAST mouse colonization susceptibility. Since all of the Collaborative Cross founder mouse strains tested have had their genomes sequenced, it was possible to compare the coding sequences of the relevant genes. It was contemplated that for a polymorphism to be relevant, it must be present in CAST but absent in other strains. No SNPs were found in MyD88, MD2 or CD14 that were unique to CAST mice. However, alignment of their TLR4 loci identified one SNP (C>T) that is unique to CAST. This SNP results in a serine to proline substitution (S312P) in the dimerization domain of CAST TLR4 (FIG. 15). This substitution did not result in reduced surface expression of TLR4 as measured by flow cytometry using anti TLR4 antibody. It was contemplated that TLR4 polymorphism may be a determinant of colonization susceptibility. TLR4^{-/-} B6 mice were inoculated with Nmus (Table 5). These TLR4^{-/-} mice are as susceptible to Nmus as MyD88^{-/-} B6 mice. C3H/HeJ mice, harboring a spontaneous mutation in TLR4, were also completely susceptible to colonization (Table 5), further supporting the argument that TLR4 signaling is involved in susceptibility to Nmus colonization.

### Example 12

### Nmus can Induce Cytokine Responses Through TLR4

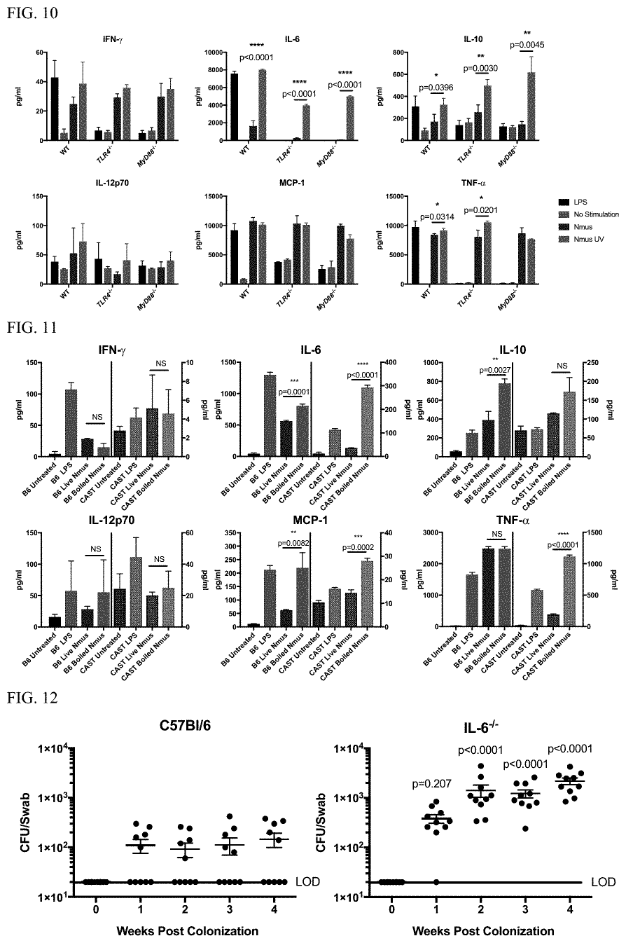

Since TLR4 and MyD88 signaling deficits correlate with Nmus colonization, the ability of Nmus to stimulate the production of cytokines by immune cells was assayed. Since Nmus is a Gram-negative bacterium, it was contemplated that its LPS would signal though TLR4. The ability of Nmus to signal through TLR4 by was tested using bone marrow derived macrophages (BMDM) from B6, TLR4^{-/-}, and MyD88^{-/-} mice. The cells were stimulated for 18 hours with live Nmus. As a positive control, cells were also stimulated with E. coli LPS. The resulting supernatants were tested for proinflammatory cytokines IFN-γ, IL-6, IL-10, IL-12p70, MCP-1, and TNF-α (FIG. 10). MyD88^{-/-} and TLR4^{-/-} cells did not respond to LPS. There was similar production of IFN-γ, MCP-1 and TNF-α in response to live Nmus by all three BMDM strains tested, indicating that production of these cytokines was independent of both TLR4 and MyD88.

It was next examined whether spleen cells would respond to ligands on the Nmus surface, or if viable bacteria were required. Spleen cells were stimulated with UV inactivated Nmus. IL-6 was produced when cells were stimulated with UV-killed but not viable Nmus, indicating live bacteria actively dampen IL-6 production. This finding is consistent with studies showing that bacterial pathogens, including Neisseria gonorrhoeae and Neisseria meningitidis, actively perturb host cell signaling during host cell contact to skew infection in their favor (Calton, C. M., et al., 2013. Cell Microbiol 15: 1837-1850). The MyD88^{-/-} and TLR4^{-/-} BMDM also produced IL-6 in response to UV-inactivated Nmus, but at a lower level than when stimulated by WT Nmus. This indicates there are TLR4/MyD88 dependent and independent signals leading to IL-6 production when stimulated with intact Nmus.

### Example 13

CAST Cells Showed Impaired Responses to Nmus and E. coli LPS

It was determined if CAST cells were impaired in their ability to respond to Nmus since this would be consistent with their ability to be colonized with Nmus. Spleen cells from CAST and B6 mice were stimulated with live Nmus, UV and heat killed Nmus, or E. coli LPS. The pattern we saw was similar to that seen in FIG. 10. B6 cells responded with IFN-γ, IL-6, IL10, IL-12, MCP-1 and TNF-α. CAST mice were severely impaired in their response to E. coli LPS as well as to live and dead Nmus (FIG. 11). Both B6 and CAST mice responded better to killed than live Nmus in their secretion of IL-6, MCP-1, IL10, IL-12 and TNF-α, although not all response differences reached statistical significance. IFN-γ did not show this regulation by live bacteria, but was produced more robustly in B6 than CAST. This enhanced response to killed Nmus indicates that there is an active suppression of the inflammatory response by living Nmus in CAST as well as in B6. The response of CAST mice to LPS and Nmus correlates with the ability to colonize, strongly supporting the idea that the defect in TLR4 mediated signaling is a major contributor to the ability to be colonized by Nmus.

### Example 14

### IL-6 Plays a Critical Role in Nmus Colonization

The cytokine, IL-6 is pivotal role in the induction of both innate and adaptive immune responses (Tanaka, T., et al., 2014. Cold Spring Harb Perspect Biol 6: a016295). Since in both CAST and B6 cells the IL-6 response was markedly different between live and killed Nmus, it was contemplated that IL-6 could play an important role in the resistance to colonization. The ability of Nmus to colonize IL-6^{-/-} mice was tested. B6-IL-6^{-/-} mice were very susceptible to colonization (10/10 colonized) and had about a five-fold higher Nmus burden compared to the B6 parental strain (Table 5 and FIG. 12). Taken together, these data indicate that inhibition of IL-6 production of by live Nmus, is a critical factor in allowing host colonization.

### Example 15

TLR4 Derived from CAST Mice is not the Cause of CAST Sensitivity to Colonization by Nmus

The experiments described above support the involvement of an innate pathway from TLR4 to MyD88 that resulted in IL-6 production. Indeed, signaling through this pathway mediated by TLR4 was dampened in CAST mice, not only when stimulated by whole Nmus and Nmus LPS, but E. coli LPS as well. The unique coding sequence polymorphism of TLR4 in CAST mice supported that an alteration of the TLR4 molecules might result in altered signaling. Consistent with this the level of surface expression of TLR4 in CAST mice was equivalent to B6, indicating that the signal transduction was altered at TLR4. The role of CAST derived TLR4 gene was tested in two ways. First, an F1 mice between TLR4^{-/-} mice and CAST was created. Secondly, mice from the Collaborative Cross with CAST TLR4 expressed on a wide variety of different genetic backgrounds were created (Aylor, D. L., et al., 2011. Genome Res 21: 1213-1222; Threadgill, D. W., et al., 2002. Mamm Genome 13: 175-178; Churchill, G. A., et al., 2004. Nat Genet 36: 1133-1137).

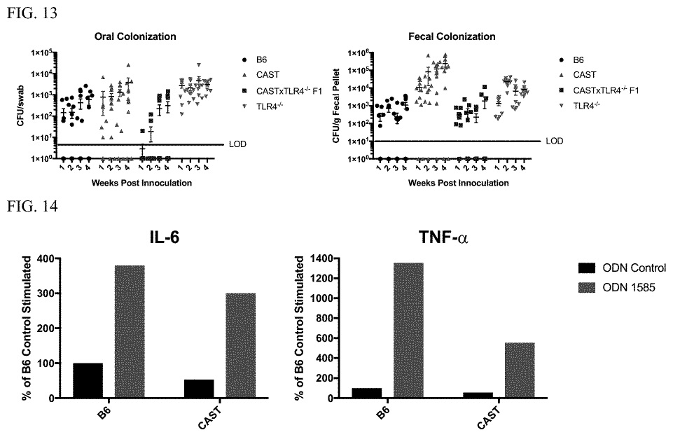

To initially test the TLR4 CAST association, CAST mice were crossed to B6-TLR4⁻/− mice. Since the TLR4^{-/-} mice, which are readily colonized, do not express any TLR4, only CAST TLR4 is expressed in these F1 animals. These mice were colonized similarly to B6 mice (4/10) consistent with signaling through TLR4^{CAST} being functional, and demonstrating the B6 background genes are able to complement the defect in TLR4 signaling defect CAST mice (FIG. 13). It remained possible that there was a gene dosage effect and the haploid dose of TLR4^{CAST} was not sufficient to allow full colonization.

In order to examine the possibility that the CAST derived TLR needed to be homozygous to display the phenotype, mice from the Collaborative Cross were tested for their ability to be colonized by Nmus. The Collaborative Cross (CC) is a set of recombinant inbred strains derived from 8 different inbred parents. It provides a unique opportunity to test the hypothesis that CAST mice polymorphism in TLR4 is responsible for susceptibility of CAST mice to Nmus colonization. CC mice whose TLR4 was either derived from CAST (designated TLR4^{CAST} here), A/J, or non-colonized parental strains were used (Ma, M., et al., 2018. Infect Immun 86). Since the CC mice are all inbred and hence homozygous for nearly every allele, one could determine if homozygous expression of the TLR4 derived from CAST mice was sufficient to allow Nmus colonization.

19 CC strains, 4 with TLR4^{CAST}, and 4 with TLR4^{A/J} (Table 6) were tested for their ability to be colonized with Nmus. Overall 10 of 19 strains were colonized. Five strains were colonized at a frequency of 50% while 4 strains were colonized at 100%. Three strains of mice that expressed TLR4^{CAST}, as well as 6 that expressed TLR4 from other founder strains, were colonized, while 1, TLR4^{CAST}, and 8 non-CAST derived TLR stains were not colonized. Strikingly, strain CC037 which did express TLR4^{CAST}, was resistant to colonization. Thus, TLR4 derived from CAST is not sufficient to allow colonization and other strains which did not express TLR4 from CAST were able to be colonized (Table 7). Since A/J mice were also colonized we repeated the analysis excluding the A/J derived TLR4 mice. In all cases tested, there was no significant association of colonization with either CAST or A/J derived TLR4. Table 6 also shows the derivation of other relevant TLR4 and MyD88 signaling genes. There was no significant association of any of these alleles with colonization.

Together these data indicate that the susceptibility of CAST and A/J colonization is not due to polymorphism of their TLR4 genes, although this does not exclude the possibility that the TLR4/MyD88 innate pathway is not involved. Rather it indicates that the genetic control is more complex than a simple alteration of the TLR4 protein.

TABLE 4

| Strain | Source |
|---|---|
| CC002/Unc | UNC- Collaborative Cross |
| CC003/Unc | UNC- Collaborative Cross |
| CC004/Tau/Unc | UNC- Collaborative Cross |
| CC006/TauUnc | UNC- Collaborative Cross |
| CC007/Unc | UNC- Collaborative Cross |
| CC009/Unc | UNC- Collaborative Cross |
| CC016/GeniUnc | UNC- Collaborative Cross |
| CC019/TauUnc | UNC- Collaborative Cross |
| CC023/GeniUnc | UNC- Collaborative Cross |
| CC037/TauUnc | UNC- Collaborative Cross |
| CC038/GeniUnc | UNC- Collaborative Cross |
| CC040TauUnc | UNC- Collaborative Cross |
| CC042GeniUnc | UNC- Collaborative Cross |
| CC045/GeniUnc | UNC- Collaborative Cross |
| CC057/Unc | UNC- Collaborative Cross |
| CC061/GeniUnc | UNC- Collaborative Cross |
| CC065/Unc | UNC- Collaborative Cross |
| CC068/TauUnc | UNC- Collaborative Cross |
| CC072/TauUnc | UNC- Collaborative Cross |
| A/J | Jackson Labs |
| C57BL/6J | Jackson Labs |
| 129S1/SvlmJ | Jackson Labs |
| NOD/ShILtJ | Jackson Labs |
| NZO/HILt/J | Jackson Labs |
| CAST/EIJ | Jackson Labs |
| PWK/PhJ | Jackson Labs |
| WSB/EIJ | Jackson Labs |
| C3H/HeJ | Jackson Labs |
| B6.129S2-ll6^{tm1Kopf}/J | Jackson Labs |
| B6.129P2(SJL)-Myd88^{tm1.1Defr}/J | Jackson Labs |
| B6.B10ScN-TIr4^{ips-del}/JthJ | Jackson Labs |

TABLE 5

| | | |
|---|---|---|
| Strain | # Colonized/Inoculated (%) | p value ⁽¹⁾ |

| | | | |
|---|---|---|---|
| C57BL/6J | 17/33 | (52) | =0.003⁽²⁾ |
| MyD88^{-/-} | 21/21 | (100) | <0.001⁽³⁾ |
| RAG-1^{-/-} | 3/14 | (21) | NS⁽³⁾ |
| TLR4^{-/-} | 24/24 | (100) | <0.001⁽³⁾ |
| C3H/HeJ | 10/10 | (100) | =0.016⁽³⁾ |
| IL-6R^{-/-} | 10/10 | (100) | =0.016⁽³⁾ |

⁽¹⁾ χ² with Yates correction for small numbers and Bonferroni tor multiple pairwise comparisons.
⁽²⁾Compared to CAST/EiJ.
⁽³⁾Compared to WT C578L/6J.
Expanded from Ma et. al. Infection and Immunity 2018

TABLE 6

| CC Number | TLR4 Ori | MD2 (Ly96) Ori | MyD88 Ori | CD14 | Colonized | Not Colonized |
|---|---|---|---|---|---|---|
| CC016/GeniUnc | A | “129” | 86 | NZO | 0 | 2 |
| CC045/GeniUnc | A | NOD | A | B6 | 0 | 2 |
| CC037/TauUnc | CAST | B6 | B6 | “129” | 0 | 5 |
| CG009/Unc | NOD | “129” | WSB | WSB | 0 | 5 |
| CC002/Unc | NZO | B6 | CAST | NZO/CAST* | 0 | 2 |
| CC003/Unc | NZO | CAST* | B6 | PWK | 0 | 2 |
| CC040TauUnc | NZO | A/CAST* | B6 | “129” | 0 | 2 |
| CC042GeniUnc | PWK | NZO | NZO | WSB* | 0 | 1 |
| CC019/TauUnc | WSB | WSB | WS8 | A | 0 | 2 |
| CC072/TauUnc | A | NZO | “129” | NZO | 1 | 1 |
| CC038/GeniUnc | CAST | NZO | “129” | NZO | 1 | 1 |
| CC065/Unc | CAST | NZO | “129” | B6 | 1 | 1 |
| CC004/Tau/Unc | NOD | “129” | NZO | PWK | 1 | 1 |
| CC007/Unc | NOD | “129” | NZO* | NOD | 1 | 1 |
| CC057/Unc | “129” | NZO | “129” | “129” | 2 | 0 |
| CC023/GeniUnc | A | “129” | PWK | “129” | 2 | 0 |
| CC068/TauUnc | CAST | A | NOD | “129” | 2 | 0 |
| CC006/TauUnc | PWK | * | CAST* | NOD | 2 | 0 |
| CC061/GeniUnc | PWK | PWK | B6 (NOD) | WSB | 2 | 0 |

TABLE 7
Effect of TLR^{CAST} on Nmus colonization in CC mice

| | Number of | Number of |
|---|---|---|
| TLR4 Genotype | Colonized mice | Resistant mice |
| CAST | 4 | 7 |
| Not CAST | 11 | 22 |

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### INCORPORATION BY REFERENCE

The entire disclosure of each of the patent documents and scientific articles referred to herein is incorporated by reference for all purposes. The following references are referenced within this application and are herein incorporated by reference in all entireties:

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

We claim:

1. A system, comprising:
a) a laboratory mouse; and
b) a strain of Neisseria musculi (Nmus)

2. The system of claim 1, wherein said Nmus is Nmus AP2365.

3. The system of claim 1, wherein said mouse is a CAST/EiJ or A/J mouse.

4. The system of claim 1, wherein the oral cavity and gut of said mouse is colonized by said Nmus.

5. The system of claim 1, wherein said system further comprises a test agent.

6. The system of claim 5, wherein said test agent is a vaccine.

7. The system of claim 5, wherein said test agent is anti-bacterial compound.

8. The system of claim 7, wherein said anti-bacterial compound targets a Neisseria gonorrhoeae (Ngo) or Neisseria meningitidis (Nme) marker.

9. The system of claim 7, wherein said anti-bacterial compound targets PilE or PilT.

10. The system of claim 7, wherein said anti-bacterial compound is a small molecule.

11. A method of screening a treatment or prevention of bacterial infection, comprising:
a) contacting the system of claim 1 with a candidate agent for treatment or prevention of a bacterial infection; and
b) determining the ability of said agent to treat or prevent said bacterial infection.

12. The method of claim 11, wherein said bacterial infection is infection by Neisseria gonorrhoeae (Ngo) or Neisseria meningitidis (Nme).

13. The method of claim 11, wherein said agent is a vaccine against said bacterial infection.

14. The method of claim 11, wherein said agent is an anti-bacterial compound.

15. The method of claim 14, wherein said anti-bacterial compound targets a Ngo or Nme marker.

16. The method of claim 14, wherein said anti-bacterial compound targets PilE or PilT.

17. The method of claim 13, wherein said anti-bacterial compound is a small molecule.

18. The method of claim 12, wherein said mouse is colonized with said Nmus prior to or after said contacting step.


SEQUENCE LISTING (3 sequences)

SEQ ID NO: 1 - DNA, 180 bp, Artificial
  Synthetic
atacattcctgtaagttacctgcatatttttccaatctgacgaacctagtacatgtggatctttcttataactatattcaaactattactgtcaacgacttacagtttctacgtgaaaatccacaagtcaatctctctttagacatgtctttgaacccaattgacttcattcaagaccaa

SEQ ID NO: 2 - DNA, 179 bp, Artificial
  Synthetic
atacattcctgtaagttacctgcatatttctccaatctgacgaacctagtacatgtggatctttcttataactatattcaaactattactgtcaacgacttacagtttctacgtgaaaatccacaagtcaatctctctttagacatatctttgaacccaatgacttcattcaagaccaa

SEQ ID NO: 3 - protein, 835 aa, Artificial
  Synthetic
MMPPWLLARTLIMALFFSCLTPGSLNPCIEVVPNITYQCMDQKLSKVPDDIPSSTKNIDLSFNPLKILKSYSFSNFSELQWLDLSRCEIETIEDKAWHGLHHLSNLILTGNPIQSFSPGSFSGLTSLENLVAVETKLASLESFPIGQLITLKKLNVAHNFIHSCKLPAYFSNLTNLVHVDLSYNYIQTITVNDLQFLRENPQVNLSLDMSLNPIDFIQDQAFQGIKLHELTLRGNFNSSNIMKTCLQNLAGLHVHRLILGEFKDERNLEIFEPSIMEGLCDVTIDEFRLTYTNDFSDDIVKFHCLANVSAMSLAGVSIKYLEDVPKHFKWQSLSIIRCQLKQFPTLDLPFLKSLTLTMNKGSISFKKVALPSLSYLDLSRNALSFSGCCSYSDLGTNSLRHLDLSFNGAIIMSANFMGLEELQHLDFQHSTLKRVTEFSAFLSLEKLLYLDISYTNTKIDFDGIFLGLTSLNTLKMAGNSFKDNTLSNVFANTTNLTFLDLSKCQLEQISWGVFDTLHRLQLLNMSHNNLLFLDSSHYNQLYSLSTLDCSFNRIETSKGILQHFPKSLAFFNLTNNSVACICEHQKFLQWVKEQKQFLVNVEQMTCATPVEMNTSLVLDFNNSTCYMYKTIISVSVVSVIVVSTVAFLIYHFYFHLILIAGCKKYSRGESIYDAFVIYSSQNEDWVRNELVKNLEEGVPRFHLCLHYRDFIPGVAIAANIIQEGFHKSRKVIVVVSRHFIQSRWCIFEYEIAQTWQFLSSRSGIIFIVLEKVEKSLLRQQVELYRLLSRNTYLEWEDNPLGRHIFWRRLKNALLDGKASNPEQTAEEEQETATWT